(19) United States
(12) Patent Application Publication
(10) Pub. No.: US 2024/0122972 A1
(43) Pub. Date: Apr. 18, 2024

(54) ANTI-PATHOGENIC LIQUID COMPOSITIONS

(71) Applicant: CLAW Biotech Holdings, LLC, St. Louis, MO (US)

(72) Inventors: Jeffery L. Dudding, Center, MO (US); Amod P. Paranjpe, Augusta, MO (US)

(21) Appl. No.: 18/277,403

(22) PCT Filed: Feb. 16, 2022
(86) PCT No.: PCT/US22/16553

§ 371 Date: Aug. 16, 2023

Related U.S. Application Data
(60) Provisional application No. 63/150,386, filed on Feb. 17, 2021.

(51) Int. Cl. A61K 33/30; A61K 9/00; A61K 9/14; A61K 33/24; A61K 33/242; A61K 33/32; A61K 33/34; A61K 33/38; A61P 31/02

(52) U.S. Cl. CPC A61K 33/30; A61K 9/0014; A61K 9/14; A61K 33/24; A61K 33/242; A61K 33/32; A61K 33/34; A61K 33/38; A61P 31/02

(57) ABSTRACT

Provided herein are anti-pathogen compositions that are suspensions or solutions (e.g., a homogeneous solution) in water (e.g., tap water or distilled water), saline, and/or a solvent comprising about 0.000000001% to about 5% by weight of an active component, wherein the active component is or comprises particles (e.g., ionic particles, microparticles, or nanoparticles) of an activated metal. The present disclosure further provides various uses for anti-pathogenic liquid compositions described herein.

### CROSS-REFERENCE TO RELATED APPLICATIONS

This application claims priority to U.S. Provisional Application No. 63/150,386, filed Feb. 17, 2021, the entirety of which is incorporated herein by reference.

### BACKGROUND

Pathogens, such as bacteria, fungi, viruses, and algae can stably exist on a dry surface or in water for hours, days, or even months. See Kramer, et al., BMC Infect. Dis., 6:130 (2016) (reviewing varying survival rates of bacteria on dry surfaces); Pinon, et al., Intervirology, 61:214-222 (2018) (reviewing survival rates for viruses in water). For example, SARS-CoV-2, the virus that causes coronavirus disease 2019 (COVID-19), which to date has infected close to a 65 million people in the United States and has killed close to one million people in the United States, is currently understood to exist for hours or even days in aerosols and on various surfaces. See van Doremalen, et al., New England J. of Med., DOI: 10.1056/NEJMc2004973 (Mar. 17, 2020), and G. Kampf, et al., J. of Hospital Infection, 104:246e251 (2020). Moreover, some pathogens can live for more than a month in water. These pathogens can cause serious infection or death.

### SUMMARY

There is a need for compositions capable of neutralizing pathogens on contact, to thereby provide control over harmful pathogens. The present disclosure encompasses a recognition that certain metals (e.g., in particular certain transition metals) are useful for neutralizing pathogens. Moreover, the present disclosure encompasses an insight that the ability of certain metals to neutralize pathogens can be increased when subjected to certain conditions. Certain metals, as described herein, after being subjected to certain conditions become “activated”, and can further be incorporated into compositions comprising, for example, water, saline, or a solvent, and retain the metal's ability to neutralize pathogens on contact. Such compositions are safe for use on every day surfaces in the home, as well as in medical facilities, manufacturing/industrial sites, commercial sites, agricultural sites, and even directly on humans (e.g., products that directly contact human skin, are inhaled, or act as surface disinfectants for commonly used household items).

The present disclosure encompasses an insight that low concentrations (e.g., lower than previously disclosed) of certain metals in solution are unexpectedly successful at neutralizing pathogens. In some embodiments, the present disclosure provides a solution or suspension (as referred to herein, “solution” and “suspension” are used interchangeably) that is anti-pathogenic, wherein the solution or suspension comprises an active component and water, saline, and/or a solvent. In some embodiments, an active component is 0.000000001% by weight to about 5% by weight of an anti-pathogenic solution. In some embodiments, an active component is 0.000000001% by weight to about 0.00001% by weight of an anti-pathogenic solution. In some embodiments, an active component is 0.00001% by weight to about 5% by weight of an anti-pathogenic solution. In some embodiments, an active component comprises particles (e.g., ionic particles, microparticles, or nanoparticles) of an activated transition metal or transition metal oxide.

Provided low-concentration compositions are particularly useful for neutralizing (i.e., inhibiting growth, replication, or otherwise killing) pathogens (e.g., bacteria, fungi, viruses, algae (e.g., cyanobacteria, dinoflagellates and diatoms), or microorganisms causing disease, in particular those that are capable of harming plants or animals (including humans or other mammals). For example, the present disclosure encompasses the insight that the compositions provided herein are capable of neutralizing many common pathogens, including methicillin resistant Staphylococcus aureus (MRSA), legionella, E. coli, and coronaviruses (e.g., SARS-CoV-2) upon contact.

### BRIEF DESCRIPTION OF DRAWINGS

FIG. 1A is an image of unactivated molybdenum particles in saline, taken by an OMAX 40×-2500× LED Digital Trinocular Microscope.

FIG. 1B is an image of unactivated molybdenum particles in polypropylene, taken by an OMAX 40×-2500× LED Digital Trinocular Microscope.

FIG. 2A is an image of activated molybdenum particles in saline, taken by an OMAX 40×-2500× LED Digital Trinocular Microscope.

FIG. 2B is an image of activated molybdenum particles in polypropylene, taken by an OMAX 40×-2500× LED Digital Trinocular Microscope.

FIG. 2C is an image of activated molybdenum powders, taken by an OMAX 40×-2500× LED Digital Trinocular Microscope.

FIG. 2D is an image of activated molybdenum powders, taken by an OMAX 40×-2500× LED Digital Trinocular Microscope.

FIG. 2E is an image of activated molybdenum powders, taken by an OMAX 40×-2500× LED Digital Trinocular Microscope.

FIG. 3 is an XRD analysis of molybdenum activated with H₂O₂.

### DETAILED DESCRIPTION

There is a need for anti-pathogen compositions capable of neutralizing pathogens on contact, both on dry surfaces (including human skin), and in water, allowing for control of harmful pathogens. The present disclosure encompasses the recognition that compositions comprising low concentrations (e.g., about 0.000000001% to about 5% by weight, about 0.00001% to about 5% by weight) of metals in an “activated” state (as described in more detail herein), are useful for neutralizing pathogens, making them invaluable for use in a variety of industries.

Activated metals useful for providing anti-pathogenic compositions are reported in PCT App. No. PCT/US20/47841, published as WO/2021/041439, the entirety of which is incorporated herein by reference in its entirety. Previous formulations reported amounts of activated metals that were greater than 0.1% in order to achieve anti-pathogenic activity. The present disclosure, however, encompasses the unexpected and surprising insight that low concentrations (e.g., lower concentrations than 0.1% by weight) of an active component can be used to achieve anti-pathogenic activity with comparable success as previously provided compositions. The present disclosure further provides insights that compositions comprising as low as about 0.000000001% by weight of an active component demonstrate anti-pathogenic activity.

Liquid Compositions

As described herein, the present disclosure provides anti-pathogen compositions that are suspensions or solutions (e.g., a homogeneous solution) in water (e.g., tap water or distilled water), saline, and/or a solvent comprising about 0.000000001% to about 5% by weight of an active component, wherein the active component is or comprises particles (e.g., ionic particles, microparticles, or nanoparticles) of an activated metal. In some embodiments, the present disclosure provides a liquid anti-pathogenic composition comprising water, saline, and/or a solvent and about 0.0000001% to about 5% by weight of an active component, wherein the active component comprises particles (e.g., ionic particles, microparticles, or nanoparticles) of an activated metal. In some embodiments, the present disclosure provides a liquid anti-pathogenic composition comprising water, saline, and/or a solvent and about 0.00001% to about 5% by weight of an active component, wherein the active component comprises particles (e.g., ionic particles, microparticles, or nanoparticles) of an activated metal.

As used herein, the term “about”, in reference to a number or percentage, is intended to include numbers that fall within a certain range around that number (where the number is real, i.e., does not go below 0% or above 100%). For example, the term “about” is intended to encompass ±0.2%, ±0.5%, ±1%, ±5%, or ±10% with respect to any indicated number.

In some embodiments, a liquid anti-pathogenic composition comprises water, saline, and/or a solvent and about 0.001% to about 1% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.001% to about 0.1% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.001% to about 0.05% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.001% to about 0.01% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.01% to about 0.1% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.01% to about 0.05% by weight of an active component, wherein the active component comprises particles (e.g., ionic particles, microparticles, or nanoparticles) of an activated metal.

In some embodiments, a liquid anti-pathogenic composition comprises water, saline, and/or a solvent and about 0.00001% to about 0.0001% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.0001% to about 0.001% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.001% to about 0.01% by weight of an active component, wherein the active component comprises particles (e.g., ionic particles, microparticles, or nanoparticles) of an activated metal.

In some embodiments, a liquid anti-pathogenic composition comprises water, saline, and/or a solvent and about 0.000000001% to about 0.0000001% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.00000001% to about 0.000001% by weight of an active component, wherein the active component comprises particles (e.g., ionic particles, microparticles, or nanoparticles) of an activated metal. In some embodiments, a liquid anti-pathogenic composition comprises water, saline, and/or a solvent and about 0.0000001% to about 0.00001% by weight of an active component, wherein the active component comprises particles (e.g., ionic particles, microparticles, or nanoparticles) of an activated metal.

In some embodiments, anti-pathogen compositions in suspension or solution in water, saline, and/or solvent neutralize pathogens upon contact. In some embodiments, such compositions are sufficiently acidic such that substantially all (e.g., 90% or greater) of pathogens are neutralized upon contact. For example, in some embodiments, an anti-pathogen liquid composition has a pH of about 5.5 or less. In some embodiments, an anti-pathogen liquid composition has a pH of about 4.0 or less. In some embodiments, an anti-pathogen liquid composition has a pH of about 3.5 or less. In some embodiments, an anti-pathogen liquid composition has a pH of about 3.0 or less. In some embodiments, an anti-pathogen liquid composition has a pH of about 2.5 or less. In some embodiments, an anti-pathogen liquid composition has a pH of about 2.0 or less. In some embodiments, an anti-pathogen liquid composition has a pH of about 1.9 or less. In some embodiments, an anti-pathogen liquid composition has a pH of about 1.85 or less. In some embodiments, an anti-pathogen liquid composition has a pH of about 1.75 or less. In some embodiments, an anti-pathogen liquid composition has a pH of about 1.65 or less. In some embodiments, an anti-pathogen liquid composition has a pH of about 1.5 or less.

In some embodiments, anti-pathogen compositions in suspension or solution in water, saline, and/or solvent neutralize pathogens upon contact. In some embodiments, such compositions have a pH of about 6 or about 7, and substantially all (e.g., 90% or greater) of pathogens are neutralized upon contact. In some embodiments, an anti-pathogen liquid composition has a pH of about 6. In some embodiments, an anti-pathogen liquid composition has a pH of about 7.

In some embodiments, anti-pathogen compositions in suspension or solution in water, saline, and/or solvent neutralize pathogens upon contact. In some embodiments, such compositions are basic such that substantially all (e.g., 90% or greater) of pathogens are neutralized upon contact. In some embodiments, an anti-pathogen liquid composition has a pH of about 7.5 or more. In some embodiments, an anti-pathogen liquid composition has a pH of about 8.0 or more. In some embodiments, an anti-pathogen liquid composition has a pH of about 8.5 or more. In some embodiments, an anti-pathogen liquid composition has a pH of about 9.0 or more. In some embodiments, an anti-pathogen liquid composition has a pH of about 9.5 or more. In some embodiments, an anti-pathogen liquid composition has a pH of about 10.0 or more. In some embodiments, an anti-pathogen liquid composition has a pH of about 10.5 or more. In some embodiments, an anti-pathogen liquid composition has a pH of about 11.0 or more. In some embodiments, an anti-pathogen liquid composition has a pH of about 11.5 or more. In some embodiments, an anti-pathogen liquid composition has a pH of about 12.0 or more. In some embodiments, an anti-pathogen liquid composition has a pH of about 12.5 or more. In some embodiments, an anti-pathogen liquid composition has a pH of about 13.0 or more. In some embodiments, an anti-pathogen liquid composition has a pH of about 13.5 or more. In some embodiments, an anti-pathogen liquid composition has a pH of about 14.0 or more.

In some embodiments, an acid is added to liquid compositions described herein to achieve a desired pH (e.g., a pH described herein). In some embodiments, an acid is glacial acetic acid. In some embodiments, a liquid composition comprises a glacial acetic acid.

In some embodiments, an active component is added to liquid compositions described herein to achieve a desired pH (e.g., a pH described herein), wherein the active component is or comprises particles (e.g., ionic particles, microparticles, or nanoparticles) of an activated metal. In some embodiments, an activated metal is or comprises at least one transition metal or transition metal oxide as described herein. In some embodiments, ZnO is added to liquid compositions described herein to achieve a desired pH (e.g., a pH described herein). In some embodiments, ZnO is added to liquid compositions described herein to achieve a pH of about 7.

In some embodiments, liquid compositions described herein are diluted to achieve a desired pH (e.g., a pH described herein). In some embodiments, a liquid composition described herein is diluted with an acid. In some embodiments, an acid is glacial acetic acid. In some embodiments, a liquid composition comprises a glacial acetic acid. In some embodiments, a liquid composition described herein is diluted with water. In some embodiments, a liquid composition described herein is diluted with saline. In some embodiments, a liquid composition described herein is diluted with a solvent.

In some embodiments, the present disclosure provides an insight that anti-pathogen compositions in suspension or solution in water, saline, and/or solvent neutralize pathogens upon contact under acidic or mildly acidic conditions (e.g., pH<7). Without being bound by theory, it is understood that, in some embodiments, the ability of anti-pathogen compositions in suspension or solution in water, saline, and/or solvent to neutralize pathogens upon contact increases as the pH decreases.

In some embodiments, the present disclosure provides an insight that anti-pathogen compositions in suspension or solution in water, saline, and/or solvent neutralize pathogens upon contact when they comprise an active component as described herein. Without being bound by theory, it is understood that, in some embodiments, the ability of anti-pathogen compositions in suspension or solution in water, saline, and/or solvent to neutralize pathogens upon contact increases as the concentration (by weight %) of an active component as described herein increases.

In some embodiments, a liquid anti-pathogenic composition provided herein comprises water and about 0.001% to about 5% by weight of an active component, wherein the active component comprises particles (e.g., ionic particles, microparticles, or nanoparticles) of an activated metal.

In some embodiments, a liquid anti-pathogenic composition provided herein comprises water and about 0.0001% to about 0.001% by weight of an active component, wherein the active component comprises particles (e.g., ionic particles, microparticles, or nanoparticles) of an activated metal.

In some embodiments, a liquid anti-pathogenic composition provided herein comprises water and about 0.000000001% to about 0.00000001% by weight of an active component, wherein the active component comprises particles (e.g., ionic particles, microparticles, or nanoparticles) of an activated metal.

In some embodiments, a liquid anti-pathogenic composition provided herein comprises a water and about 0.00000001% to about 0.0000001% by weight of an active component, wherein the active component comprises particles (e.g., ionic particles, microparticles, or nanoparticles) of an activated metal.

In some embodiments, a liquid anti-pathogenic composition provided herein comprises a water and about 0.0000001% to about 0.000001% by weight of an active component, wherein the active component comprises particles (e.g., ionic particles, microparticles, or nanoparticles) of an activated metal.

In some embodiments, a liquid anti-pathogenic composition provided herein comprises a water and about 0.000001% to about 0.00001% by weight of an active component, wherein the active component comprises particles (e.g., ionic particles, microparticles, or nanoparticles) of an activated metal.

In some embodiments, a liquid anti-pathogenic composition provided herein comprises saline and about 0.001% to about 5% by weight of an active component, wherein the active component comprises particles (e.g., ionic particles, microparticles, or nanoparticles) of an activated metal. Saline, as used herein, refers to a liquid mixture of water and salt. In some embodiments, saline is a saturated solution. In some embodiments, saline is supersaturated.

In some embodiments, a liquid anti-pathogenic composition provided herein comprises saline and about 0.00001% to about 0.001% by weight of an active component, wherein the active component comprises particles (e.g., ionic particles, microparticles, or nanoparticles) of an activated metal. Saline, as used herein, refers to a liquid mixture of water and salt. In some embodiments, saline is a saturated solution. In some embodiments, saline is supersaturated.

In some embodiments, a liquid anti-pathogenic composition provided herein comprises saline and about 0.000000001% to about 0.00000001% by weight of an active component, wherein the active component comprises particles (e.g., ionic particles, microparticles, or nanoparticles) of an activated metal. In some embodiments, saline is supersaturated.

In some embodiments, a liquid anti-pathogenic composition provided herein comprises saline and about 0.00000001% to about 0.0000001% by weight of an active component, wherein the active component comprises particles (e.g., ionic particles, microparticles, or nanoparticles) of an activated metal. In some embodiments, saline is supersaturated.

In some embodiments, a liquid anti-pathogenic composition provided herein comprises saline and about 0.0000001% to about 0.000001% by weight of an active component, wherein the active component comprises particles (e.g., ionic particles, microparticles, or nanoparticles) of an activated metal. In some embodiments, saline is supersaturated.

In some embodiments, a liquid anti-pathogenic composition provided herein comprises saline and about 0.000001% to about 0.00001% by weight of an active component, wherein the active component comprises particles (e.g., ionic particles, microparticles, or nanoparticles) of an activated metal. In some embodiments, saline is supersaturated.

In some embodiments, a liquid anti-pathogenic composition provided herein comprises a solvent and about 0.001% to about 5% by weight of an active component, wherein the active component comprises particles (e.g., ionic particles, microparticles, or nanoparticles) of an activated metal. In some embodiments, a solvent is or comprises an alcohol (e.g., methanol, ethanol, n-propanol, isopropyl alcohol, and the like).

In some embodiments, a liquid anti-pathogenic composition provided herein comprises a solvent and about 0.00001% to about 0.001% by weight of an active component, wherein the active component comprises particles (e.g., ionic particles, microparticles, or nanoparticles) of an activated metal. In some embodiments, a solvent is or comprises an alcohol (e.g., methanol, ethanol, n-propanol, isopropyl alcohol, and the like).

In some embodiments, a liquid anti-pathogenic composition provided herein comprises a solvent and about 0.000000001% to about 0.00000001% by weight of an active component, wherein the active component comprises particles (e.g., ionic particles, microparticles, or nanoparticles) of an activated metal. In some embodiments, a solvent is or comprises an alcohol (e.g., methanol, ethanol, n-propanol, isopropyl alcohol, and the like).

In some embodiments, a liquid anti-pathogenic composition provided herein comprises a solvent and about 0.00000001% to about 0.0000001% by weight of an active component, wherein the active component comprises particles (e.g., ionic particles, microparticles, or nanoparticles) of an activated metal. In some embodiments, a solvent is or comprises an alcohol (e.g., methanol, ethanol, n-propanol, isopropyl alcohol, and the like).

In some embodiments, a liquid anti-pathogenic composition provided herein comprises a solvent and about 0.0000001% to about 0.000001% by weight of an active component, wherein the active component comprises particles (e.g., ionic particles, microparticles, or nanoparticles) of an activated metal. In some embodiments, a solvent is or comprises an alcohol (e.g., methanol, ethanol, n-propanol, isopropyl alcohol, and the like).

In some embodiments, a liquid anti-pathogenic composition provided herein comprises a solvent and about 0.000001% to about 0.00001% by weight of an active component, wherein the active component comprises particles (e.g., ionic particles, microparticles, or nanoparticles) of an activated metal. In some embodiments, a solvent is or comprises an alcohol (e.g., methanol, ethanol, n-propanol, isopropyl alcohol, and the like).

In some embodiments, a liquid anti-pathogenic composition is a lotion, oil, ointment, or other formulation suitable for topical delivery. In some embodiments, a liquid anti-pathogenic composition further comprises excipients useful for topical formulations, including, for example, waxes, emollients, thickening agents/viscosity increasing agents, humectants, pH modifiers, water repelling agents, anti-foaming agents, surfactants, solubilizers, wetting agents, penetration enhancers, and antioxidants.

Activated Metals

In some embodiments of compositions provided herein, an activated metal is any transition metal or oxide thereof. As used herein, an “oxide” of a transition metal refers to a transition metal that has been oxidized, i.e., the metal is in a cationic form and, in some embodiments, has bound to one or more counterions (e.g., chalcogens, such as oxygen or sulfur) to stabilize the cationic form of the metal. Exemplary transition metals that are useful in embodiments described herein include Mn, Mo, Zn, Cu, Au, and Ag, as well as their known oxidized forms (e.g., Mn(VII), Mn(VI), Mn(V), Mn(IV), Mn(III), Mn(II), Mn(I), Mo(IV), Mo(V), Mo(VI), Zn(II), Cu(I), Cu(II), Au(I), Au(III), Ag(I), MnO, Mn₃O₄, Mn₂O₃, MnO₂, MnO₃, Mn₂O₇, H₂MnO₄, HMnO₄, MoO₂, MoO₃, Mo₂O₆, H₂MoO₅, ZnO, Cu₂O, CuO, Au₂O, Au₂O₃, and Ag₂O).

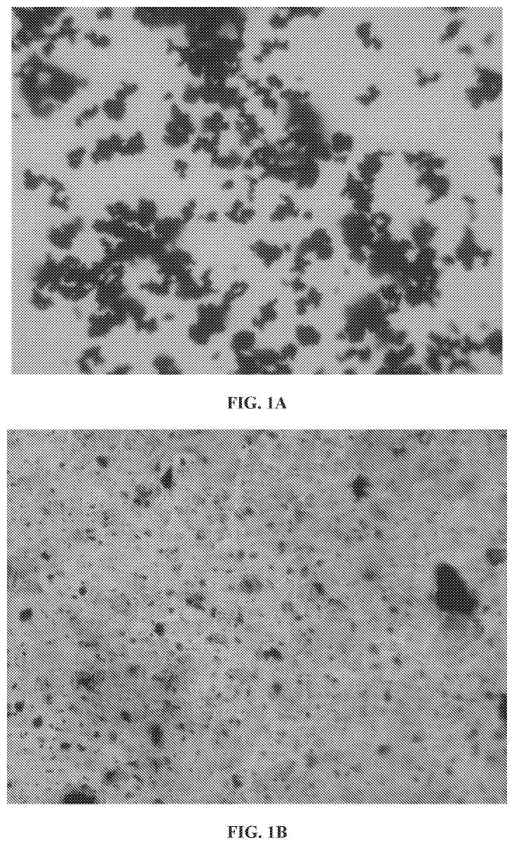

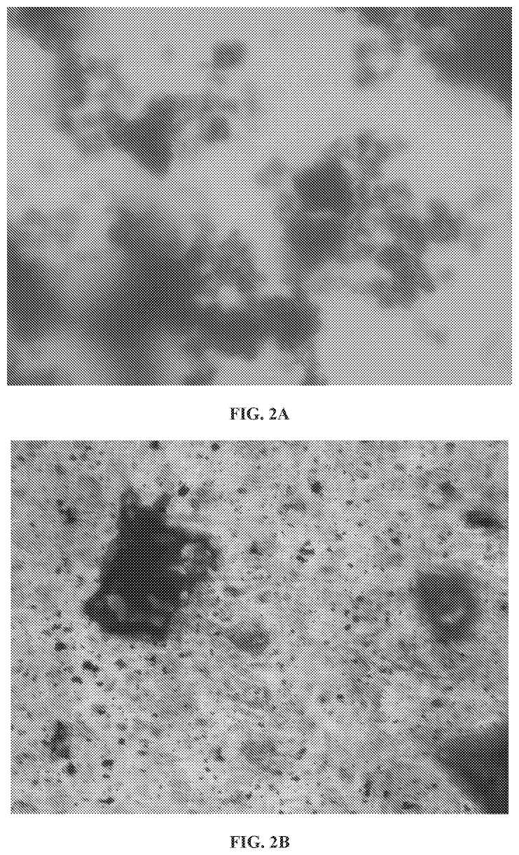

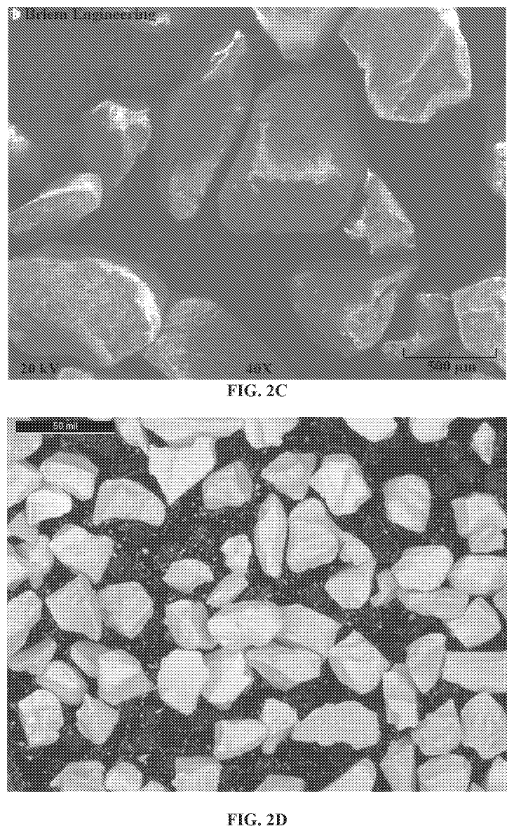

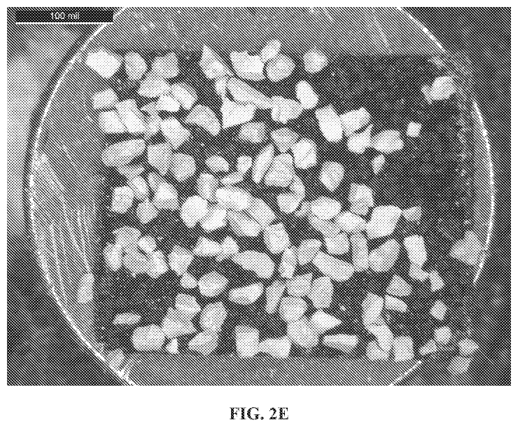

Compositions provided in the present disclosure utilize an activated form of the metals described herein. An “activated” metal (i.e., a metal that is in an activated state or form), as used herein, refers to a metal that has been subjected to certain conditions and/or otherwise achieves a state demonstrated (in the present disclosure) to increase anti-pathogen activity. Such activated metals have a different conformation than then metal atom prior to activation. For example, as illustrated in the examples below, molybdenum, prior to activation take a shape as seen in FIGS. 1A and 1B. After being subjected to activation conditions, however, molybdenum takes a shape as seen in FIGS. 2A, 2B, 2C, 2D, and 2E. Once in the form as seen in FIG. 2A, 2B, 2C, 2D, or 2E, the activated molybdenum is more effective at neutralizing pathogens when exposed either in exposed in a liquid form (i.e., when the pathogen is exposed to the molybdenum in a liquid solution or suspension). The activation process described herein further improves the anti-pathogen properties of metals already in oxidized form.

As described herein, in some embodiments, the present disclosure provides anti-pathogen compositions comprising an active component, wherein the active component comprises an activated metal, and wherein an activated metal is or comprises at least one transition metal or transition metal oxide. In some embodiments, at least one transition metal or transition metal oxide is selected from Mn, Mo, Zn, Cu, Au, Ag, or an oxide thereof. In some embodiments, at least one transition metal or transition metal oxide is selected from Cu, Au, Ag, or an oxide thereof. In some embodiments, at least one transition metal or transition metal oxide is selected from Mo, Zn, or an oxide thereof. In some embodiments, at least one transition metal or transition metal oxide is Mn or an oxide thereof. In some embodiments, at least one transition metal or transition metal oxide is Mo or an oxide thereof. In some embodiments, at least one transition metal or transition metal oxide is Zn or an oxide thereof. In some embodiments, at least one transition metal or transition metal oxide is Cu or an oxide thereof. In some embodiments, at least one transition metal or transition metal oxide is Au or an oxide thereof. In some embodiments, at least one transition metal or transition metal oxide is Ag or an oxide thereof.

In some embodiments, at least one transition metal or transition metal oxide is Mn(VII), Mn(VI), Mn(V), Mn(IV), Mn(III), Mn(II), Mn(I), Mo(IV), Mo(V), Mo(VI), Zn(II), Cu(I), Cu(II), Au(I), Au(III), or Ag(I). In some embodiments, at least one transition metal or transition metal oxide is Cu(I), Cu(II), Au(I), Au(III), or Ag(I). In some embodiments, at least one transition metal or transition metal oxide is Mo(IV), Mo(V), Mo(VI), or Zn(II). In some embodiments, at least one transition metal or transition metal oxide is Mo(IV), Mo(V), or Mo(VI). In some embodiments, at least one transition metal or transition metal oxide is Mo(IV). In some embodiments, at least one transition metal or transition metal oxide is Mo(V). In some embodiments, at least one transition metal or transition metal oxide is Mo(VI). In some embodiments, at least one transition metal or transition metal oxide is Mn(VII), Mn(VI), Mn(V), Mn(IV), Mn(III), Mn(II), Mn(I). In some embodiments, at least one transition metal or transition metal oxide is Mn(VII). In some embodiments, at least one transition metal or transition metal oxide is Mn(VI). In some embodiments, at least one transition metal or transition metal oxide is Mn(V). In some embodiments, at least one transition metal or transition metal oxide is Mn(IV). In some embodiments, at least one transition metal or transition metal oxide is Mn(III). In some embodiments, at least one transition metal or transition metal oxide is Mn(II). In some embodiments, at least one transition metal or transition metal oxide is Mn(I). In some embodiments, at least one transition metal or transition metal oxide is Zn(II). In some embodiments, at least one transition metal or transition metal oxide is Cu(I) or Cu(II). In some embodiments, at least one transition metal or transition metal oxide is Cu(I). In some embodiments, at least one transition metal or transition metal oxide is Cu(II). In some embodiments, at least one transition metal or transition metal oxide is Au(I) or Au(III). In some embodiments, at least one transition metal or transition metal oxide is Au(I). In some embodiments, at least one transition metal or transition metal oxide is Au(III). In some embodiments, at least one transition metal or transition metal oxide is Ag(I).

In some embodiments, at least one transition metal or transition metal oxide is Mn, MnO, Mn₃O₄, Mn₂O₃, MnO₂, MnO₃, Mn₂O₇, H₂MnO₄, HMnO₄, Mo, MoO₂, MoO₃, Mo₂O₆, H₂MoO₅, Zn, ZnO, Cu, Cu₂O, CuO, Au, Au₂O, Au₂O₃, Ag, or Ag₂O. In some embodiments, at least one transition metal or transition metal oxide is Cu, Cu₂O, CuO, Au, Au₂O, Au₂O₃, Ag, or Ag₂O. In some embodiments, at least one transition metal or transition metal oxide is Mo, MoO₂, MoO₃, Mo₂O₆, H₂MoO₅, Zn, or ZnO. In some embodiments, at least one transition metal or transition metal oxide is Mn, Mn₂O₃, MnO₂, MnO₃, Mn₂O₇. In some embodiments, at least one transition metal or transition metal oxide is MnO or Mn₃O₄. In some embodiments, at least one transition metal or transition metal oxide is Mn, In some embodiments, at least one transition metal or transition metal oxide is MnO. In some embodiments, at least one transition metal or transition metal oxide is Mn₃O₄. In some embodiments, at least one transition metal or transition metal oxide is Mn₂O₃. In some embodiments, at least one transition metal or transition metal oxide is MnO₂. In some embodiments, at least one transition metal or transition metal oxide is MnO₃. In some embodiments, at least one transition metal or transition metal oxide is Mn₂O₇. In some embodiments, at least one transition metal or transition metal oxide is Mo, MoO₂, or MoO₃. In some embodiments, at least one transition metal or transition metal oxide is Mo or MoO₃. In some embodiments, at least one transition metal or transition metal oxide is Mo. In some embodiments, at least one transition metal or transition metal oxide is MoO₂. In some embodiments, at least one transition metal or transition metal oxide is MoO₃. In some embodiments, at least one transition metal or transition metal oxide is Zn or ZnO. In some embodiments, at least one transition metal or transition metal oxide is Zn. In some embodiments, at least one transition metal or transition metal oxide is ZnO. In some embodiments, at least one transition metal or transition metal oxide is Cu, Cu₂O, or CuO. In some embodiments, at least one transition metal or transition metal oxide is Cu or CuO. In some embodiments, at least one transition metal or transition metal oxide is Cu. In some embodiments, at least one transition metal or transition metal oxide is Cu₂O. In some embodiments, at least one transition metal or transition metal oxide is CuO. In some embodiments, at least one transition metal or transition metal oxide is Au, Au₂O, or Au₂O₃. In some embodiments, at least one transition metal or transition metal oxide is Au or Au₂O₃. In some embodiments, at least one transition metal or transition metal oxide is Au. In some embodiments, at least one transition metal or transition metal oxide is Au₂O. In some embodiments, at least one transition metal or transition metal oxide is Au₂O₃. In some embodiments, at least one transition metal or transition metal oxide is Ag or Ag₂O. In some embodiments, at least one transition metal or transition metal oxide is Ag. In some embodiments, at least one transition metal or transition metal oxide is Ag₂O.

As described herein, a transition metal or transition metal oxide, once activated, can change crystal structure as compared to the unactivated form. For example, in some embodiments, a transition metal oxide can have an orthorhombic crystal structure. In some embodiments, at least one activated metal is Mo, or an oxide thereof, having an orthorhombic crystal structure.

The activated metals described herein can be in particle form (e.g., a microparticle or a nanoparticle). As used herein, a “microparticle” is a particle that is between 1 and 1000 μm in size. As used herein, a “nanoparticle” is a particle that is between 1 and 1000 nm in size.

In some embodiments, particles of at least one active metal are microparticles having a size of about 1 μm to about 1000 μm. In some embodiments, particles of at least one active metal are microparticles having a size of about 45 μm to about 1000 μm. In some embodiments, particles of at least one active metal are microparticles having a size of about 50 μm to about 1000 μm. In some embodiments, particles of at least one active metal are microparticles having a size of about 75 μm to about 1000 μm. In some embodiments, particles of at least one active metal are microparticles having a size of about 100 μm to about 1000 μm. In some embodiments, particles of at least one active metal are microparticles having a size of about 1 μm to about 100 μm. In some embodiments, particles of at least one active metal are microparticles having a size of about 10 μm to about 85 μm. In some embodiments, particles of at least one active metal are microparticles having a size of about 10 μm to about 50 μm. In some embodiments, particles of at least one active metal are microparticles having a size of about 20 μm to about 50 μm. In some embodiments, particles of at least one active metal are microparticles having a size of about 30 μm to about 50 μm. In some embodiments, particles of at least one active metal are microparticles having a size of about 40 μm to about 50 μm. In some embodiments, particles of at least one active metal are microparticles having a size of about 40 μm to about 45 μm. In some embodiments, particles of at least one active metal are microparticles having a size of about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, or about 50 μm.

In some embodiments, particles of at least one active metal are nanoparticles having a size of about 1 nm to about 1000 nm. In some embodiments, particles of at least one active metal are nanoparticles having a size of about 500 nm to about 1000 nm. In some embodiments, particles of at least one active metal are nanoparticles having a size of about 1 nm to about 500 nm. In some embodiments, particles of at least one active metal are nanoparticles having a size of about 1 nm to about 100 nm. In some embodiments, particles of at least one active metal are nanoparticles having a size of about 10 nm to about 85 nm. In some embodiments, particles of at least one active metal are nanoparticles having a size of about 10 nm to about 50 nm. In some embodiments, particles of at least one active metal are nanoparticles having a size of about 20 nm to about 50 nm. In some embodiments, particles of at least one active metal are nanoparticles having a size of about 30 nm to about 50 nm. In some embodiments, particles of at least one active metal are nanoparticles having a size of about 40 nm to about 50 nm. In some embodiments, particles of at least one active metal are nanoparticles having a size of about 40 nm to about 45 nm. In some embodiments, particles of at least one active metal are nanoparticles having a size of about 30 nm, about 31 nm, about 32 nm, about 33 nm, about 34 nm, about 35 nm, about 36 nm, about 37 nm, about 38 nm, about 39 nm, about 40 nm, about 41 nm, about 42 nm, about 43 nm, about 44 nm, about 45 nm, about 46 nm, about 47 nm, about 48 nm, about 49 nm, or about 50 nm.

In some embodiments, at least one active metal (e.g., molybdenum) is or comprises an ionic form of the metal (e.g., ionic molybdenum). For example, in some embodiments, Mo loses electron(s) (e.g., becomes positively charged, for example, is a cationic molybdenum ion) or oxidizes once active and thereby becomes ionic, which makes Mo a cation in the formation of an ionic bond with a negatively charged anion (for example, with a non-metal anion). In some embodiments, an active metal has a charge state (i.e., an oxidation state) that is +1, +2, +3, +4, +5, +6, or +7. In some embodiments, at least one active metal is molybdenum having a charge state that is +2, +3, +4, +5, or +6. In some embodiments, at least one active metal is molybdenum having a charge state that is +2, +4, or +6. In some embodiments, at least one active metal is molybdenum having a charge state that is +2. In some embodiments, at least one active metal is molybdenum having a charge state that is +4. In some embodiments, at least one active metal is molybdenum having a charge state that is +6. In some embodiments, at least one active metal is molybdenum having a charge state that is +7. In some embodiments, at least one active metal is manganese having a charge state that is +2, +3, +4, +5, +6, or +7. In some embodiments, at least one active metal is manganese having a charge state that is +4, +6, or +7. In some embodiments, at least one active metal is manganese having a charge state that is +4. In some embodiments, at least one active metal is manganese having a charge state that is +6. In some embodiments, at least one active metal is manganese having a charge state that is +7.

It is understood that, as described herein, an active metal having a charge state can either be dissociated (e.g., be ionic in a solution), or associated with one or more suitable counterions. For example, molybdenum having a +4 charge state useful in embodiments described herein can be in the form of Mo⁺⁴ as a dissociated ion, or, when associated with one or more counterions, could be in the form of MoO₂, H₂MoO₅, including hydrates thereof. A person of skill in the art would understand suitable counterions useful for creating a chemically stable active metal for various charge states of metals reported herein.

The compositions of the present disclosure can further comprise a second metal or metal oxide. For example, in some embodiments, an anti-pathogenic liquid composition provided herein further comprises a second metal selected from Ni, Zn, Mn, Cu, Au, Ag, Sn, and Pd, or oxides thereof. In some embodiments, a second metal is Ni. In some embodiments, a second metal is Pd. In some embodiments, a second metal is Sn. In some embodiments, a second metal is Ag. In some embodiments, a second metal is Au. In some embodiments, a second metal is Cu. In some embodiments, a second metal is Mn. In some embodiments, a second metal is Zn or ZnO. In some embodiments, a second metal is Zn. In some embodiments, a second metal is ZnO.

In some embodiments, an anti-pathogen liquid composition comprises about 0.001% to about 5% by weight of a second metal. In some embodiments, an anti-pathogen liquid composition comprises about 0.001% to about 1% by weight of the second metal. In some embodiments, an anti-pathogen liquid composition comprises about 0.001% to about 0.05% by weight of the second metal. In some embodiments, an anti-pathogen liquid composition comprises about 0.001% to about 0.01% by weight of the second metal. In some embodiments, an anti-pathogen liquid composition comprises about 0.001% to about 0.005% by weight of the second metal.

In some embodiments, a liquid anti-pathogenic composition provided herein comprises water, saline and/or solvent and about 0.00001% to about 0.0001% by weight of a second metal. In some embodiments, a liquid anti-pathogenic composition provided herein comprises water, saline, and/or a solvent and about 0.0001% to about 0.001% by weight of a second metal.

In some embodiments, a liquid anti-pathogenic composition provided herein comprises water, saline and/or solvent and about 0.000000001% to about 0.00000001% by weight of a second metal. In some embodiments, a liquid anti-pathogenic composition provided herein comprises water, saline and/or solvent and about 0.00000001% to about 0.0000001% by weight of a second metal. In some embodiments, a liquid anti-pathogenic composition provided herein comprises water, saline and/or solvent and about 0.0000001% to about 0.000001% by weight of a second metal. In some embodiments, a liquid anti-pathogenic composition provided herein comprises water, saline and/or solvent and about 0.000001% to about 0.00001% by weight of a second metal.

Anti-pathogen liquid compositions described herein are useful in a variety of sanitization methods, including, for example, as anti-pathogens for solid surfaces (e.g., as an aerosol and/or as a spray delivered from a spray bottle, a mist, a fogger, and the like that can be sprayed or applied to a solid surface). In some embodiments, the present disclosure provides a method for neutralizing microbes or pathogens on a surface, the method comprising a step of contacting the surface with an anti-pathogen liquid composition described herein. In some embodiments, the surface is human skin.

In some embodiments, a liquid composition described herein can be used in combination with a nebulizer to form an aerosol spray. In some embodiments, the aerosol spray can be used to neutralize pathogens on solid surfaces.

In some embodiments, a liquid composition described herein can be used as a spray, e.g., to be sprayed onto a solid surface via a spray bottle, a mist, a foggers, and the like.

In some embodiments, a microbe or pathogen is selected from a micrococcus, staphylococcus, bacillus, pseudomonas, legionella, salmonella, listeria, Clostridium perfringens, Acinetobacter baumannii, Escherichia coli, coronaviruses, rhinoviruses, influenza, adenovirus, parainfluenza, respiratory syncytial virus, and enterovirus. In some embodiments, a microbe or pathogen is a staphylococcus (including, e.g., methicillin-Staphylococcus aureus (MRSA)), legionella, an influenza, E. coli, or a coronavirus (including SARS-CoV-2).

Assessment and/or Characterization

In some embodiments, an activated metal, and/or a composition including it, may be characterized and/or assessed for one or more features as described herein.

For example, in some embodiments, ability to sanitize may be assessed. In some embodiments, ability to sanitize may be or include ability to inhibit proliferation of and/or to kill one or more microbes or pathogens as described herein (e.g., micrococcus, staphylococcus, bacillus, pseudomonas, legionella, salmonella, listeria, Clostridium perfringens, Acinetobacter baumannii, Escherichia coli, coronaviruses, rhinoviruses, influenza, adenovirus, parainfluenza, respiratory syncytial virus, and enterovirus).

In some embodiments, ability to sanitize may be assessed with respect to direct contact—e.g., ability of an activated metal and/or composition as described herein to reduce proliferation and/or to kill one or more microbes or pathogens when contacted with a sample including such microbe(s) or pathogens. Alternatively or additionally, in some embodiments, ability to sanitize may be assessed over a distance—e.g., ability of an activated metal and/or composition as described herein to reduce proliferation and/or to kill one or more microbes or pathogens in a space or area notwithstanding that the activated metal and/or composition may not be in direct contact with the microbe or pathogen.

Activation of Metals

The present disclosure also provides methods for activating a metal, e.g., a transition metal or transition metal oxide, as described herein. In some embodiments a method for activating a transition metal or transition metal oxide comprises treating the transition metal or transition metal oxide with one or more of heating, calcination, washing/oxidizing, charging, UV light exposure, and combinations thereof.

For example, in some embodiments, a transition metal or transition metal oxide is activated by exposing the transition metal or transition metal oxide to a temperature of 100° C.-2400° C. for a period of time, e.g., 10 minutes to 24 hours.

In some embodiments, a transition metal or transition metal oxide is activated by exposing the transition metal or transition metal oxide to a wash. In some embodiments, the wash is an aqueous oxidation agent. In some embodiments, the wash fluid is a gaseous oxidation agent. In some embodiments, the wash fluid consists of 1-4 parts H₂O, 1-4 parts distilled H₂O, 1-35% H₂O₂ (peroxide), acetylene, oxyacetylene, or combinations thereof.

In some embodiments, a transition metal or transition metal oxide is activated by exposing the transition metal or transition metal oxide to low voltage. The ideal ranges of the voltage and duration of the charging may vary depending on the individual components and aggregate. This pretreatment causes the components to have a specific charge at their surface to further disable and/or kill pathogens.

In some embodiments, a transition metal or transition metal oxide is activated by exposing the transition metal or transition metal oxide to UV light. In some embodiments, the UV light is selected from UVA, UVB, UVC, and combinations thereof. Increased anti-pathogen efficacy has been observed using UV light for zinc oxide nanoparticles and titanium dioxide doped with molybdenum. This pretreatment results in a photocatalytic effect from the active component. The photocatalytic effect is particularly helpful in aqueous and dark environments. In some embodiments, the photocatalytic effect may be used in the brewing industry.

In some embodiments, a metal is activated after being incorporated into any of the liquid compositions described herein. For example, in some embodiments, a liquid suspension comprises a transition metal or transition metal oxide, which is then subjected to the activation conditions described herein (e.g., washing/oxidizing, calcination, heating, charging, UV light exposure, and combinations thereof).

In some embodiments, an ionic activated metal refers to metal atoms having a charge state (i.e., a cationic charge) of +1, +2, +3, +4, +5, +6, or +7. For example, an ionic activated metal is or comprises molybdenum particles (e.g., nanoparticles and the like) that have a cationic charge, (e.g., +1, +2, +3, +4, +5, or +6). In some embodiments, an ionic activated metal is or comprises molybdenum particles (e.g., nanoparticles and the like) that are activated (e.g., by exposure to H₂O₂).

In some embodiments, an ionic activated metal can be prepared according to methods provided herein. For example, in some embodiments, a method of activating a metal in an ionic form comprises contacting a metal with an oxidizing agent. In some embodiments, the present disclosure provides a method of activating a metal comprising contacting the metal with hydrogen peroxide. In some embodiments, a metal to be activated is described herein, and includes, for example, Mn, Mo, Zn, Cu, Ag, and Au. In some embodiments, a metal to be activated is Mo. In some embodiments, a metal to be activated is Mn. In some embodiments, the present disclosure provides a metal is activated by contact with H₂O₂.

For example, in some embodiments, the washing process may result in sediment, which may be collected and used to activate saline. In some embodiments, methods of producing the at least one active metal in an ionic form may further comprise conducting one or more tests on an activated saline using filters having a size of about 1 μm. In some embodiments, sediment having a size of at least about 1 μm, which may comprise microparticles and/or ionic forms may be trapped in the filters. In some embodiments, sediment having a size of 0 to about 1 μm (e.g., nanoparticles) may pass through the filters.

Uses

The present disclosure also provides uses of anti-pathogen liquid compositions described herein. In some embodiments, an anti-pathogen liquid composition is used in medical facilities, manufacturing/industrial sites, commercial sites, agricultural sites, and even directly on humans (e.g., products that directly contact human skin, are inhaled, or act as surface disinfectants for commonly used household items).

In some embodiments, an anti-pathogen liquid composition is applied to a food item as a spray. In some embodiment, a food item is a fruit. In some embodiment, a food item is a vegetable.

In some embodiments, an anti-pathogen liquid composition is applied to a household item as a spray. In some embodiments, a household item is a food container. In some embodiments, a household item is a food processing item. In some embodiments, a household item is a food display. In some embodiments, a household item is a toilet. In some embodiments, a household item is a shower or shower equipment. In some embodiments, a household item is a humidifier.

In some embodiments, an anti-pathogen liquid composition is applied to human skin. In some embodiment, an anti-pathogen liquid composition is applied to human skin prior to a surgical procedure. In some embodiment, an anti-pathogen liquid composition is applied to human skin during a surgical procedure. In some embodiment, an anti-pathogen liquid composition is applied to human skin after a surgical procedure.

### Exemplary Embodiments

The embodiments presented below are examples of compositions, methods and uses described in the present application.

Embodiment 1. An anti-pathogenic liquid composition comprising:
about 0.000000001% by weight to about 5% by weight of particles of an active component; and
water, saline, and/or a solvent,
wherein the active component is or comprises at least one activated metal.

Embodiment 2. An anti-pathogenic liquid composition comprising:
about 0.00001% by weight to about 5% by weight of particles of an active component; and
water, saline, and/or a solvent,
wherein the active component is or comprises at least one activated metal.

Embodiment 3. The anti-pathogenic liquid composition of Embodiment 2, wherein the composition comprises about 0.001% by weight to about 1% by weight of particles of an active component.

Embodiment 4. The anti-pathogenic liquid composition of Embodiments 2 or 3, wherein the composition comprises about 0.001% by weight to about 0.1% by weight of particles of an active component.

Embodiment 5. The anti-pathogenic liquid composition of any one of Embodiments 1-4, wherein the composition comprises about 0.001% by weight to about 0.05% by weight of particles of an active component.

Embodiment 6. The anti-pathogenic liquid composition of any one of Embodiments 1-5, wherein the composition comprises about 0.001% by weight to about 0.01% by weight of particles of an active component.

Embodiment 7. The anti-pathogenic liquid composition of Embodiment 1, wherein the composition comprises about 0.00001% by weight to about 0.001% by weight of particles of an active component.

Embodiment 8. The anti-pathogenic liquid composition of Embodiment 1, wherein the composition comprises about 0.000000001% by weight to about 0.001% by weight of particles of an active component.

Embodiment 9. The anti-pathogenic liquid composition of Embodiment 1, 6, or 8, wherein the composition comprises about 0.00001% by weight to about 0.0001% by weight of particles of an active component.

Embodiment 10. The anti-pathogenic liquid composition of Embodiment 1, 6, or 8, wherein the composition comprises about 0.0001% by weight to about 0.001% by weight of particles of an active component.

Embodiment 11. The anti-pathogenic liquid composition of Embodiment 1, 6, or 8, wherein the composition comprises about 0.000000001% by weight to about 0.00000001% by weight of particles of an active component.

Embodiment 12. The anti-pathogenic liquid composition of Embodiment 1, 6, or 8, wherein the composition comprises about 0.00000001% by weight to about 0.0000001% by weight of particles of an active component.

Embodiment 13. The anti-pathogenic liquid composition of Embodiment 1, 6, or 8, wherein the composition comprises about 0.0000001% by weight to about 0.000001% by weight of particles of an active component.

Embodiment 14. The anti-pathogenic liquid composition of Embodiment 1, 6, or 8, wherein the composition comprises about 0.000001% by weight to about 0.00001% by weight of particles of an active component.

Embodiment 15. The anti-pathogenic liquid composition of any one of Embodiments 1-14, wherein the at least one activated metal is a transition metal or a transition metal oxide.

Embodiment 16. The anti-pathogenic liquid composition of any one of Embodiments 1-15, wherein the at least one activated metal is selected from Mn, Mo, Zn, Cu, Au, Ag, or an oxide thereof.

Embodiment 17. The anti-pathogenic liquid composition of Embodiment 16, wherein the at least one activated metal is Mo, Mo(IV), Mo(V) or Mo(VI), or an oxide thereof.

Embodiment 18. The anti-pathogenic liquid composition of Embodiment 16, wherein the at least one activated metal is Mn, Mn(VII), Mn(VI), Mn(V), Mn(IV), Mn(III), Mn(II), Mn(I), or an oxide thereof.

Embodiment 19 The anti-pathogenic liquid composition of Embodiment 16, wherein the at least one activated metal or oxide thereof is selected from Mn, MnO, Mn₃O₄, Mn₂O₃, MnO₂, MnO₃, Mn₂O₇, H₂MnO₄, HMnO₄, Mo, MoO₂, MoO₃, MoO₅, Mo₂O₆, H₂MoO₅, Zn, ZnO, Cu, Cu₂O, CuO, Au, AuO, Au₂O₃, Ag, and Ag₂O.

Embodiment 20. The anti-pathogenic liquid composition Embodiment 19, wherein the at least one activated metal or oxide thereof is Mo, MoO₂, MoO₃, H₂MoO₅, or Mo₂O₆.

Embodiment 21. The anti-pathogenic liquid composition Embodiment 19, wherein the at least one activated metal or oxide thereof is Mn, MnO, Mn₃O₄, Mn₂O₃, MnO₂, MnO₃, Mn₂O₇, H₂MnO₄, HMnO₄.

Embodiment 22. The anti-pathogenic liquid composition of any one of Embodiments 1-21, wherein the at least one activated metal is Mo, or an oxide thereof, having a cubic, spherical, monoclinic, hexagonal, orthorhombic, tetragonal, triclinic, or rhombohedral crystal structure.

Embodiment 23. The anti-pathogenic liquid composition of any one of Embodiments 1-22, wherein the pH of the anti-pathogenic liquid composition is about 5.5 or less.

Embodiment 24. The anti-pathogenic liquid composition of any one of Embodiments 1-23, wherein the pH of the anti-pathogenic liquid composition is about 4.0 or less.

Embodiment 25. The anti-pathogenic liquid composition of any one of Embodiments 1-24, wherein the pH of the anti-pathogenic liquid composition is about 2.0 or less.

Embodiment 26. The anti-pathogenic liquid composition of any one of Embodiments 1-25, wherein the pH of the anti-pathogenic liquid composition is about 1.5 or less.

Embodiment 27. The anti-pathogenic liquid composition of any one of Embodiments 1-22, wherein the pH of the anti-pathogenic liquid composition is about 7.

Embodiment 28. The anti-pathogenic liquid composition of any one of Embodiments 1-22, wherein the pH of the anti-pathogenic liquid composition is about 7.5 or more.

Embodiment 29. The anti-pathogenic liquid composition of any one of Embodiments 1-22 and 28, wherein the pH of the anti-pathogenic liquid composition is about 9 or more.

Embodiment 30. The anti-pathogenic liquid composition of any one of Embodiments 1-22, 28, and 29, wherein the pH of the anti-pathogenic liquid composition is about 11.5 or more.

Embodiment 31. The anti-pathogenic liquid composition of any one of Embodiments 1-22 and 28-30, wherein the pH of the anti-pathogenic liquid composition is about 14.

Embodiment 32. The anti-pathogenic liquid composition of any one of Embodiments 1-31, wherein the particles of the at least one active metal are microparticles, wherein the microparticles have a size of about 1 μm to about 1000 μm.

Embodiment 33. The anti-pathogenic liquid composition of any one of Embodiments 1-32, wherein the particles of the at least one active metal are microparticles, wherein the microparticles have a size of about 10 μm to about 85 μm.

Embodiment 34. The anti-pathogenic liquid composition of any one of Embodiments 1-33, wherein the particles of the at least one active metal are microparticles, wherein the microparticles have a size of about 10 μm to about 50 μm.

Embodiment 35. The anti-pathogenic liquid composition of any one of Embodiments 1-34, wherein the particles of the at least one active metal are microparticles, wherein the microparticles have a size of about 20 μm to about 50 μm.

Embodiment 36. The anti-pathogenic liquid composition of any one of Embodiments 1-35, wherein the particles of the at least one active metal are microparticles, wherein the microparticles have a size of about 30 μm to about 50 μm.

Embodiment 37. The anti-pathogen liquid composition of any one of Embodiments 1-36, wherein the particles of the at least one active metal are microparticles, wherein the microparticles have a size of about 40 μm to about 50 μm.

Embodiment 38. The anti-pathogenic liquid composition of any one of Embodiments 1-37, wherein the particles of the at least one active metal are microparticles, wherein the microparticles have a size of about 40 μm to about 45 μm.

Embodiment 39. The anti-pathogenic liquid composition of any one of Embodiments 1-31, wherein the particles of the at least one active metal are nanoparticles, wherein the nanoparticles have a size of about 1 nm to about 1000 nm.

Embodiment 40. The anti-pathogenic liquid composition of any one of Embodiments 1-31 and 39, wherein the particles of the at least one active metal are nanoparticles, wherein the nanoparticles have a size of about 10 nm to about 85 nm.

Embodiment 41. The anti-pathogenic liquid composition of any one of Embodiments 1-31, 39, and 40, wherein the particles of the at least one active metal are nanoparticles, wherein the nanoparticles have a size of about 10 nm to about 50 nm.

Embodiment 42. The anti-pathogenic liquid composition of any one of Embodiments 1-31 and 39-41, wherein the particles of the at least one active metal are nanoparticles, wherein the nanoparticles have a size of about 20 nm to about 50 nm.

Embodiment 43. The anti-pathogenic liquid composition of any one of Embodiments 1-31 and 39-42, wherein the particles of the at least one active metal are nanoparticles, wherein the nanoparticles have a size of about 30 nm to about 50 nm.

Embodiment 44. The anti-pathogen liquid composition of any one of Embodiments 1-31 and 39-43, wherein the particles of the at least one active metal are nanoparticles, wherein the nanoparticles have a size of about 40 nm to about 50 nm.

Embodiment 45. The anti-pathogenic liquid composition of any one of Embodiments 1-31 and 39-43, wherein the particles of the at least one active metal are nanoparticles, wherein the nanoparticles have a size of about 40 nm to about 45 nm.

Embodiment 46. The anti-pathogenic liquid composition of any one of Embodiments 1-45, further comprising a second metal or metal oxide.

Embodiment 47. The anti-pathogenic liquid composition of Embodiment 46, wherein the second metal is selected from Ni, Zn, Mn, Au, Ag, Cu, and Pd, or oxides thereof.

Embodiment 48. The anti-pathogenic liquid composition of Embodiments 46 or 47, wherein the second metal is selected from Zn or ZnO.

Embodiment 49. The anti-pathogenic liquid composition of any one of Embodiments 46-48, wherein the anti-pathogenic liquid composition comprises about 0.000000001% to about 5% by weight of the second metal.

Embodiment 50. The anti-pathogenic liquid composition of any one of Embodiments 46-49, wherein the anti-pathogenic liquid composition comprises about 0.1% to about 5% by weight of the second metal.

Embodiment 51. The anti-pathogenic liquid composition of any one of Embodiments 46-50, wherein the anti-pathogenic liquid composition comprises about 0.1% to about 3% by weight of the second metal.

Embodiment 52. The anti-pathogenic liquid composition of any one of Embodiments 46-51, wherein the anti-pathogenic liquid composition comprises about 0.1% to about 1% by weight of the second metal.

Embodiment 53. The anti-pathogenic liquid composition of any one of Embodiments 46-49, wherein the anti-pathogenic liquid composition comprises about 0.00001% to about 0.001% by weight of the second metal.

Embodiment 54. The anti-pathogenic liquid composition of any one of Embodiments 46-49, wherein the anti-pathogenic liquid composition comprises about 0.001% to about 0.1% by weight of the second metal.

Embodiment 55. The anti-pathogenic liquid composition of any one of Embodiments 46-49, wherein the anti-pathogenic liquid composition comprises about 0.000000001% to about 0.00000001% by weight of the second metal.

Embodiment 56. The anti-pathogenic liquid composition of any one of Embodiments 46-49, wherein the anti-pathogenic liquid composition comprises about 0.00000001% to about 0.0000001% by weight of the second metal.

Embodiment 57. The anti-pathogenic liquid composition of any one of Embodiments 46-49, wherein the anti-pathogenic liquid composition comprises about 0.000001% to about 0.00001% by weight of the second metal.

Embodiment 58. The anti-pathogenic liquid composition of any one of Embodiments 46-49, wherein the anti-pathogenic liquid composition comprises about 0.00001% to about 0.0001% by weight of the second metal.

Embodiment 59. A method for neutralizing pathogens on a surface, the method comprising a step of contacting the surface with the anti-pathogenic liquid composition of any one of Embodiments 1-58.

Embodiment 60. The method of claim 59, wherein the surface is human skin.

Embodiment 61. The method of Embodiment 60, wherein the step of contacting human skin occurs prior to a surgical procedure.

Embodiment 62. The method of Embodiment 60, wherein the step of contacting human skin occurs during a surgical procedure.

Embodiment 63. The method of Embodiment 60, wherein the step of contacting human skin occurs after a surgical procedure.

Embodiment 64. The method of Embodiments 59-63, wherein the pathogens are selected from Gram positive bacteria, Gram negative bacteria, fungi, viruses, and algae.

Embodiment 65. The method of any one of Embodiments 59-63, wherein the pathogens are selected from micrococcus, staphylococcus, bacillus, pseudomonas, legionella, salmonella, listeria, Clostridium perfringens, Acinetobacter baumannii, Escherichia coli, coronaviruses, rhinoviruses, influenza, norovirus, adenovirus, parainfluenza, respiratory syncytial virus, and enterovirus.

### Exemplification

The present teachings include descriptions provided in the Examples that are not intended to limit the scope of any claim. The following non-limiting examples are provided to further illustrate the present teachings. Those of skill in the art, in light of the present application, will appreciate that many changes can be made in the specific embodiments that are provided herein and still obtain a like or similar result without departing from the spirit and scope of the present teachings.

### Example 1—Activation of Metals and Metal Oxides

Metals described in these examples are activated as described in the present application and in PCT App. No. PCT/US20/47841, published as WO/2021/041439, which is incorporated herein by reference in its entirety.

Activation by H₂O₂ (“Washing”)

Mo particles of having a size of about 40 μm to about 45 μm were submerged in an aqueous solution of about 35% H₂O₂. Metal particles started with black/gray coloration and changed to yellow coloration after a period of time. The particles were then filtered and vacuum dried before being used in experiments or further incorporated into suspensions or solutions, as described below.

A change in structure of the activated Mo particles was visualized on an OMAX 40×-2500× LED Digital Trinocular Microscope. An image of unactivated Mo particles under microscope is seen in FIGS. 1A and 1B. An image of activated Mo particles under microscope is seen in FIGS. 2A, 2B, and 2C. It is observed that Mo particles, after activation, take on an orthorhombic structure.

### Activation by Calcination

MoO₃ particles having a size of 44 μm were heated at 250° C. for two hours. The resulting particles were allowed to cool and then used in experiments directly or further incorporated into suspensions or solutions, as described below.

### Example 2—Preparation of Anti-Pathogenic Composition

Mo particles from Example 1, having a size of about 40 μm to about 45 μm are submerged in an aqueous solution of about 35% H₂O₂. Metal particles start with black/gray coloration and chang to yellow coloration after a period of time. The particles are then filtered and the filtrate is obtained.

The filtrate contains low concentration of activated Mo, e.g., Mo, MoO₂, MoO₃, Mo₂O₆, H₂MoO₅. In some embodiments, the concentration of activated Mo is about 0.000000001%.

The filtrate resulting from the process of the present Example demonstrates anti-pathogenic activities.

### Example 3—Effectiveness of Metal Suspension Against SARS-CoV-2 and Coronavirus 229E

A saline solution comprising SARS-CoV-2 or Coronavirus 229E is prepared according to standard methods (Spray 1A and Spray 1B, respectively)). A suspension comprising Mo particles (40-45 μm particle size) activated by washing in saline and SARS-CoV-2 or Coronavirus 229E are prepared (Spray 2A and Spray 2B, respectively).

A polypropylene surface and a surface prepared as described in Example 6 will each be sprayed with each of Sprays 1A-2B. Each surface will be tested to determine the amount of SARS-CoV-2 and Coronavirus-229E remaining on each surface at each of 1 hour, 3 hours, and 6 hours.

### Example 4—Stability of Activated Metal Suspensions

Samples of activated Mo and MoO₃ in suspension were found to be shelf stable for at least six months. The following samples were stored at room temperature under ambient conditions, and exhibited consistent pH range after six months.

| Sample | Metal | Concentration/Ratio | pH |
|---|---|---|---|
| 1 | Activated MoO₃ | 2.5 g metal/ | 2.2 |
| | | 100 mL saline | |
| 2 | Activated Mo | 1 g metal/ | 1.95 |
| | | 100 mL saline | |
| 3 | Activated Mo | 5 g metal/ | 1.4 |
| | | 100 mL saline | |
| 4 | Unactivated Mo | 1 g metal/ | 4 |
| | | 100 mL saline | |

A dry mixture of having a 1:1 ratio by weight of activated Mo to ZnO was stored for six months at room temperature under ambient conditions. After six months, no signs of degradation evidenced by color change were observed.

### Example 5—Surface Time-Kill Test of Stainless Steel Coupons Sprayed with Saline Solution Treated with Molybdenum Against MS-2 Bacteriophage (Virus) and Human Coronavirus Strain 229E

### Example 5a—MS-2 Bacteriophage (Virus)

MS-2 is a non-enveloped RNA virus and used as a surrogate for a large range of human enteric pathogenic viruses (e.g., enteroviruses, noroviruses, rotaviruses, and hepatitis A and E viruses).

### Materials

Prior to experimentation, 25 g of activated molybdenum powder (>99% pure; 45 μm particle size) was added to 250 ml of sterile phosphate buffered saline (PBS; pH 7.4) to create a 10% molybdenum solution (w/v) in a sterile Erlenmeyer flask. This solution was mixed thoroughly and then allowed to settle at room temperature for approximately 90 hours. Following this, the supernatant was carefully removed from the flask by pipetting while ensuring that the settled powder was not disturbed. This supernatant was used as the treated saline solution for the experiment and was stored at room temperature until use.

The experiment was conducted on 2″×2″ stainless steel coupons sprayed with the 10% molybdenum solution in PBS. The coupons were inoculated with 0.1 ml of MS-2 stock solution containing approximately 5.0×10⁸ plaque forming units (PFU)/ml of MS-2 bacteriophage. MS-2 is a non-pathogenic virus that infects E. coli and other members of the family Enterobacteriaceae. It is commonly used as a human enteric virus surrogate because it is similar in size and shape and exhibits comparable resistance to various disinfectants. The virus inoculum was spread over the entire surface of the coupon using a sterile pipet tip. Duplicate coupons were included for each exposure contact time for both the test samples (sprayed with PBS containing molybdenum) and the control samples (sprayed with PBS only).

Immediately following inoculation, the coupons were sprayed once using a spray bottle from a distance of approximately 6 inches with either the molybdenum-treated PBS (test samples) or PBS alone (control samples).

The inoculated and sprayed coupons were placed in sealed Tupperware chambers with moist paper towels and incubated at room temperature (22.1° C.) to prevent drying which would lead to a reduction in viral numbers.

Duplicate samples from the control coupons were collected immediately upon inoculation to determine the baseline viral concentration recovered at t=0 minutes. The coupons were sampled by thorough rinsing with 1 ml of Dey-Engley (D/E) neutralizing broth in a sterile petri dish. The rinse solution was collected and placed into sterile 1.5 ml Eppendorf tubes.

All other control and test samples were held at room temperature for the remainder of the experiment (22.1° C. at a relative humidity of ˜95%). At t=1, 5, 15, and 30 minutes, duplicate samples of the remaining control and test coupons were sampled and treated in the manner described previously.

To quantify the numbers of recovered viable MS-2 from each coupon, serial 10-fold dilutions of the neutralized samples were performed in sterile PBS and 0.1-ml volumes of each dilution were assayed using the double-agar overlay technique with duplicate plates for each dilution. In short, approximately 0.5 ml of a log-phase culture (3-4 hours growth in tryptic soy broth medium with agitation at 37° C.) of host Escherichia coli bacterium were added to 5 ml of molten tryptic soy agar (containing 1% agar) in a test tube. Next, 0.1 ml of each dilution of the test sample was added to the tube. The tubes were then vortexed gently to mix the cultures and poured onto the surfaces of separate tryptic soy agar plates. The plates were swirled gently to cover the entire surface of the plate with the agar overlay. The overlay was then allowed to solidify at room temperature and then the plates were incubated (inverted) for 18 to 24 hours at 37° C. The surviving MS-2 were enumerated by counting plaques (circular clearings in the bacterial growth on the agar overlays) to determine the number of PFU of virus per milliliter of each sample.

In order to confirm that the antimicrobial solution was sufficiently neutralized by the D/E, a neutralization verification test was performed. A volume of 0.4 ml of the 10% molybdenum in PBS solution was placed into 1 ml of D/E neutralizing broth. The solution was mixed and then MS-2 was added to a final concentration of approximately 5.2×10⁷ PFU. The solution was mixed again and then was allowed to sit for ten minutes at room temperature (22.1° C.). Ten-fold serial dilutions of the neutralized solution were assayed as described previously. If the solution was completely neutralized, it was expected that there would be no reduction in MS-2 numbers in comparison to the controls in PBS alone.

The data were reported as the logarithmic reduction using the formula −log₁₀ (Nₜ/N₀), where N₀ is the concentration of the recovered MS-2 at time=0 minutes and Nₜ is the concentration of the viable MS-2 in the sample collected at time=t (i.e., 1, 5, 15, or 30 minutes). The percent reduction was also calculated.

A Student's t-test was used to statistically compare the reductions observed with the test spray containing molybdenum with the reductions observed with the control PBS spray. The reductions were considered to be statistically significant if the resultant P value was <0.05.

### Results

No reduction in MS-2 was observed during the neutralization verification test, indicating that the D/E was successful in neutralizing the 10% molybdenum in PBS solution.

Inoculum=5.2×10⁷ PFU/coupon

Number of viable MS-2 virus particles recovered per coupon:
Control Sample A (0 minutes)=3.65×10⁷ PFU
Control Sample B (0 minutes)=3.66×10⁷ PFU
Control Sample A (1 minute)=4.35×10⁷ PFU
Control Sample B (1 minute)=4.10×10⁷ PFU
Control Sample A (5 minutes)=2.51×10⁷ PFU
Control Sample B (5 minutes)=3.90×10⁷ PFU
Control Sample A (15 minutes)=3.25×10⁷ PFU
Control Sample B (15 minutes)=3.75×10⁷ PFU
Control Sample A (30 minutes)=3.7×10⁷ PFU
Control Sample B (30 minutes)=4.10×10⁷ PFU
Test Sample A (1 minute)=2.61×10⁷ PFU
Test Sample B (1 minute)=9.65×10⁶ PFU
Test Sample A (5 minutes)=2.29×10⁷ PFU
Test Sample B (5 minutes)=2.01×10⁷ PFU
Test Sample A (15 minutes)=2.85×10⁶ PFU
Test Sample B (15 minutes)=1.77×10⁷ PFU
Test Sample A (30 minutes)=1.86×10⁶ PFU
Test Sample B (30 minutes)=4.50×10⁶ PFU
Geometric mean number of viable MS-2 bacteriophage recovered from control spray samples after 1 minute=4.22×10⁷ PFU/coupon.
Geometric mean number of viable MS-2 bacteriophage recovered from control spray samples after 5 minutes=3.13×10⁷ PFU/coupon.
Geometric mean number of viable MS-2 bacteriophage recovered from control spray samples after 15 minutes=3.49×10⁷ PFU/coupon.
Geometric mean number of viable MS-2 bacteriophage recovered from control spray samples after 30 minutes=3.89×10⁷ PFU/coupon.
Geometric mean number of viable MS-2 bacteriophage recovered from test spray samples after 1 minute=1.59×10⁷ PFU/coupon.
Geometric mean number of viable MS-2 bacteriophage recovered from test spray samples after 5 minutes=2.15×10⁷ PFU/coupon.
Geometric mean number of viable MS-2 bacteriophage recovered from test spray samples after 15 minutes=7.10×10⁶ PFU/coupon.
Geometric mean number of viable MS-2 bacteriophage recovered from test spray samples after 30 minutes=2.89×10⁶ PFU/coupon.
Percent reduction from test spray samples after 1 minute=57.3%.
Percent reduction from test spray samples after 5 minutes=41.1%.
Percent reduction from test spray samples after 15 minutes=80.5%.
Percent reduction from test spray samples after 30 minutes=92.1%.

The table below shows antimicrobial efficacy of test samples sprayed with PBS with or without 10% activated molybdenum (Mo) with a contact time of either 1, 5, 15, or 30 minutes against MS-2 bacteriophage. Experiment was conducted with duplicate samples at 22.1° C. at a relative humidity of ˜95% (t=0 minutes collected immediately following inoculation).

| | | Spray Treatment | | | | | |
|---|---|---|---|---|---|---|---|
| | | Control coupons (PBS) | | | Test flasks (PBS w/10% Mo) | | |
| | | | Average | | | Average | |
| Exposure | | | Log₁₀ | Average | | Log₁₀ | Average |
| Time | | Log₁₀ | Reduction ± | Percent | Log₁₀ | Reduction ± | Percent |
| (Minutes) | Sample | Reduction * | SD | Reduction | Reduction * | SD | Reduction |
| 1 | A | 0.00 | 0.00 ± | 0.0 | 0.15 | 0.37 ± | 57.3 |
| | B | 0.00 | 0.00 | | 0.58 | 0.30 | |
| 5 | A | 0.16 | 0.08 ± | 16.8 | 0.20 | 0.23 ± | 41.1 |
| | B | 0.00 | 0.11 | | 0.26 | 0.04 | |
| 15 | A | 0.05 | 0.03 ± | 6.7 | 1.11 | 0.71 ± | 80.5 |
| | B | 0.00 | 0.04 | | 0.31 | 0.57 | |
| 30 | A | 0.00 | 0.00 ± | 0.0 | 1.29 | 1.10 ± | 92.1 |
| | B | 0.00 | 0.00 | | 0.91 | 0.27 | |

* Initial concentration of MS-2 bacteriophage was approximately 5.20 × 10⁷ PFU/coupon; an average of 3.65 × 10⁷ PFU was recovered from the control coupons at t = 0 minutes. This value was used to calculate the log₁₀ reductions for the subsequent samples collected.

### SD Standard Deviation

### Discussion

The concentrations of MS-2 recovered from the control samples remained consistent over the entire course of the experiment. In contrast, small reductions in viable MS-2 were observed within 1 minute on the samples treated with the 10% molybdenum spray solution. The reductions observed after 1, 5, and 15 minutes of contact time were not statistically significant in comparison to the control samples (P≤0.05). However, within 30 minutes of contact time, the reductions in MS-2 recovered (average reduction of 1.10 log₁₀) were statistically significant in comparison to the control samples (P=0.029).

### Example 5b—Surface Time-Kill Test of Stainless Steel Coupons Sprayed with Saline Solution Treated with Molybdenum Against Human Coronavirus Strain 229E

### Materials

Prior to the experiment, 25 g of activated molybdenum powder (>99% pure; 45 μm particle size) was added to 250 ml of sterile phosphate buffered saline (PBS; pH 7.4) to create a 10% molybdenum solution (w/v) in a sterile Erlenmeyer flask. This solution was mixed thoroughly and then allowed to settle at room temperature for approximately 90 hours. Following this, the supernatant was carefully removed from the flask by pipetting while ensuring that the settled powder was not disturbed. This supernatant was used as the treated saline solution for the experiment and was stored at room temperature until use.

The experiment was conducted on 2″×2″ stainless steel coupons sprayed with a 10% molybdenum solution in PBS. The coupons were inoculated with 0.1 ml of virus stock solution containing approximately 1.0×10⁶ TCID₅₀/ml of human coronavirus strain 229E. The inoculum was spread over the entire surface of the coupon using a sterile pipet tip. Duplicate coupons were included for each exposure contact time for both the test samples (sprayed with PBS containing molybdenum) and the control samples (sprayed with PBS only).

Immediately following inoculation, the coupons were sprayed twice using a spray bottle from a distance of approximately 6 inches with either the molybdenum-treated PBS (test samples) or PBS alone (control samples).

The inoculated and sprayed coupons were placed in sealed Tupperware chambers with moist paper towels and incubated at room temperature (22.3° C.) to prevent drying which would lead to a large reduction in viral numbers.

Duplicate samples from the control coupons were collected immediately upon inoculation to determine the baseline viral concentration recovered at t=0 minutes. The coupons were sampled by thorough rinsing with 1 ml of Dey-Engley (D/E) neutralizing broth in a sterile petri dish. The rinse solution was collected and placed into sterile 1.5 ml Eppendorf tubes.

The collected samples were passed through Sephadex gel filtration columns to reduce cytotoxicity in the subsequent cell culture assay. Following this, the samples were passed through separate syringe filters (0.45 m pore size; pre-wetted with 3% beef extract to prevent virus adsorption to the filters) to remove any contaminants such as bacteria or fungi. This step was necessary since the experiment was not conducted in a sterile environment.

All other control and test samples were held at room temperature for the remainder of the experiment (22.3° C. at a relative humidity of ˜95%). At t=1, 5, 15, and 30 minutes, duplicate samples of the remaining control and test coupons were sampled and treated in the manner described previously.

Virus concentrations for each neutralized and filtered sample were quantified using the Reed-Muench method to determine the tissue culture infectious dose that affected 50% of the wells (TCID₅₀). The samples were 10-fold serially diluted in minimal essential media (MEM). The assay was performed in 96-well cell culture plates containing monolayers of MRC-5 cells (fetal human lung fibroblast. Prior to the assay, the MRC-5 cells were gently rinsed twice with MEM and then the 96-well plates were inoculated with the diluted samples (6 wells inoculated with 50 microliters each per dilution) and the plates were incubated in an atmosphere of 5% CO2 for 1 hour at 35° C. to allow the virus particles to adsorb to the cells.

Each 96-well plate also included at least 6 negative control wells containing cells only (no antimicrobials or virus) with 50 microliters of MEM added.)

Following this incubation period, 150 microliters of MEM containing 2% fetal bovine serum was added to each of the 96 wells and the plates were incubated in an atmosphere of 5% CO₂ for 6 days at 35° C.

The cells were observed daily for viral cytopathic effects (CPE) using an inverted microscope. The inoculated cells were compared to the negative control cells in the same 96-well plate to differentiate CPE from un-inoculated cells. Any CPE that was observed within 24 hours of incubation was considered to be caused by cytotoxicity (caused by sensitivity of the cells to the D/E neutralizing buffer or the antimicrobial) since CPE caused by coronavirus typically requires ≥2 days. Wells positive for CPE following 2 or more days were considered positive for viral growth. No CPE was observed in any of the negative control wells.)

After the incubation period, the TCID₅₀/coupon was determined. Six wells per dilution were used to ensure adequate precision of the assay. The greatest dilution in which 50% or higher of the wells were positive was used to determine the virus TCID₅₀/coupon following the method described by Payment and Trudel.

In order to confirm that the antimicrobial solution was sufficiently neutralized by the D/E, a neutralization verification test was performed. A volume of 0.4 ml of the 10% molybdenum in PBS solution (the estimated volume found in one spray) was placed into 1 ml of D/E neutralizing broth. The solution was mixed and then human coronavirus 229E was added to a final concentration of approximately 1.0×10⁶ TCID₅₀. The solution was mixed again and then was allowed to sit for ten minutes at room temperature (22.3° C.). Ten-fold serial dilutions of the neutralized solution were assayed as described previously. If the solution was completely neutralized, it was expected that there would be no reduction in coronavirus 229E numbers in comparison to the controls in PBS alone.

The data were reported as the logarithmic reduction using the formula −log₁₀ (Nₜ/N₀), where N₀ is the concentration of the recovered coronavirus at time=0 minutes and N t is the concentration of the surviving coronavirus in the sample collected at time=t (i.e., 1, 5, 15, or 30 minutes). The percent reduction was also calculated.

A Student's t-test was used to statistically compare the reductions observed with the test spray containing molybdenum with the reductions observed with the control PBS spray. The reductions were considered to be statistically significant if the resultant P value was <0.05.

### Results

Inoculum=˜1.0×10⁶TCID₅₀/coupon

Number of viable virus particles recovered per coupon:
Control Sample A (0 minutes)=3.6×10⁵ TCID₅₀
Control Sample B (0 minutes)=1.1×10⁶ TCID₅₀
Control Sample A (1 minute)=3.6×10⁵ TCID₅₀
Control Sample B (1 minute)=5.0×10⁵ TCID₅₀
Control Sample A (5 minutes)=4.3×10⁵ TCID₅₀
Control Sample B (5 minutes)=9.3×10⁵ TCID₅₀
Control Sample A (15 minutes)=3.6×10⁵ TCID₅₀
Control Sample B (15 minutes)=4.3×10⁵ TCID₅₀
Control Sample A (30 minutes)=4.3×10⁵ TCID₅₀
Control Sample B (30 minutes)=2.0×10⁵ TCID₅₀
Test Sample A (1 minute)=3.6×10⁵ TCID₅₀
Test Sample B (1 minute)=3.6×10⁵ TCID₅₀
Test Sample A (5 minutes)=2.0×10⁵ TCID₅₀
Test Sample B (5 minutes)=4.3×10⁴ TCID₅₀
Test Sample A (15 minutes)=2.0×10⁴ TCID₅₀
Test Sample B (15 minutes)=4.3×10⁴ TCID₅₀
Test Sample A (30 minutes)=1.1×10⁴ TCID₅₀
Test Sample B (30 minutes)=2.0×10⁴ TCID₅₀
Geometric mean number of viable human coronavirus 229E recovered from control spray samples after 1 minute=4.3×10⁵ TCID₅₀/coupon
Geometric mean number of viable human coronavirus 229E recovered from control spray samples after 5 minutes=6.3×10⁵ TCID₅₀/coupon
Geometric mean number of viable human coronavirus 229E recovered from control spray samples after 15 minutes=3.9×10⁵ TCID₅₀/coupon
Geometric mean number of viable human coronavirus 229E recovered from control spray samples after 30 minutes=3.0×10⁵ TCID₅₀/coupon
Geometric mean number of viable human coronavirus 229E recovered from test spray samples after 1 minute=3.6×10⁵ TCID₅₀/coupon
Geometric mean number of viable human coronavirus 229E recovered from test spray samples after 5 minutes=9.3×10⁴ TCID₅₀/coupon
Geometric mean number of viable human coronavirus 229E recovered from test spray samples after 15 minutes=3.0×10⁴ TCID₅₀/coupon
Geometric mean number of viable human coronavirus 229E recovered from test spray samples after 30 minutes=1.5×10⁴ TCID₅₀/coupon
Percent reduction from test spray samples after 1 minute=30.8%
Percent reduction from test spray samples after 5 minutes=82.2%
Percent reduction from test spray samples after 15 minutes=94.4%
Percent reduction from test spray samples after 30 minutes=97.1%

The table below shows antimicrobial efficacy of test samples sprayed with PBS with or without 10% activated molybdenum (Mo) with a contact time of either 1, 5, 15, or 30 minutes against human coronavirus strain 229E (ATCC #VR-740). Experiment was conducted with duplicate samples at 22.3° C. at a relative humidity of ˜95% (t=0 hours collected immediately following inoculation).

| | | Log₁₀ removal* | | | |
|---|---|---|---|---|---|
| Treatment | Sample | 1 min | 5 min | 15 min | 30 min |
| Control | Sample 1 | 0.16 | 0.08 | 0.16 | 0.08 |
| Spray | Sample 2 | 0.01 | 0.00 | 0.08 | 0.41 |
| (PBS) | Average (±SD) | 0.09 ± 0.11 | 0.04 ± 0.06 | 0.12 ± 0.06 | 0.25 ± 0.24 |
| Test Spray | Sample 1 | 0.16 | 0.41 | 1.41 | 1.66 |
| (PBS w/ | Sample 2 | 0.16 | 1.08 | 1.08 | 1.41 |
| 10% Mo) | Average (±SD) | 0.16 ± 0.00 | 0.75 ± 0.47 | ^{†}1.25 ± 0.24 | ^{†}1.54 ± 0.18 |

*Initial concentration of human coronavirus 229E was approximately 1.0 × 10⁶ TCID₅₀/coupon; an average of 5.1 × 10⁵ TCID₅₀ was recovered from the control coupons at t = 0 minutes. This value was used to calculate the log₁₀ reductions for the subsequent samples collected.
SD Standard deviation.
^{†}Reduction was statistically significant (P ≤ 0.05) in comparison to the reductions observed on the control stainless steel coupons at the same exposure contact time.

### Discussion

A loss of approximately 0.29 log₁₀ was observed between the number of virus particles inoculated onto the coupons and the number of particles recovered from the control samples at t=0 minutes.

No reductions in human coronavirus 229E were observed during the neutralization verification test, indicating that the D/E was successful in neutralizing the 10% molybdenum in PBS solution.

The PBS solution containing 10% activated molybdenum was effective at reducing the numbers of viable human coronavirus 229E particles after 15 and 30 minutes of contact time (1.25 and 1.54 log₁₀, respectively). The observed reductions were statistically significant in comparison to the reductions observed in the control samples after 15 and 30 minutes (P=0.023 and 0.025, respectively), but not after 1 and 5 minutes of contact time (P=0.42 and 0.17, respectively).

### Example 6—Suspension Time-Kill Test of Molybdenum Powder Suspended in Phosphate Buffered Saline Against Acinetobacter baumannii and Candida albicans

### Example 6a—Suspension Time-Kill Test of Molybdenum Powder Suspended in Phosphate Buffered Saline Against Acinetobacter baumannii

### Methods

A culture of Acinetobacter baumannii was prepared on the day before testing by inoculating one colony of the test organism into 100 ml of tryptic soy broth (TSB) and incubation overnight at 37° C.

On the test date, the bacterial cells were washed by pelleting the cells via centrifugation. The supernatant was discarded and the pellet was re-suspended in 0.01 M phosphate-buffered saline (PBS; pH 7.4). Three washing steps were performed in total.

The cell suspension was diluted in 100 ml of sterile PBS in 250-ml screw cap Erlenmeyer flasks to obtain a density of ˜1×10⁵ colony-forming units (CFU) per ml. Six flasks were included in the experiment (3 control flasks, 3 test flasks).

Samples from the three control flasks were collected immediately upon inoculation/mixing to determine the baseline bacterial concentration at t=0 hours. A volume of 0.1 ml was removed from each and placed into separate 0.9-ml volumes of Dey-Engley (D/E) neutralizing broth. The samples were vortexed for 10 seconds and then 10-fold serially diluted in PBS. The various dilutions were inoculated onto tryptic soy agar (TSA) plates using the spread plate method. The plates were incubated for 24 to 48 hours at 37° C. and the colonies enumerated.

Also at t=0, 2 grams of pure molybdenum powder was added to each of the test flasks and the solution mixed thoroughly to result in a 2% Mo solution (wt %). All six flasks were placed on an orbital shaker at room temperature 20.8° C. with agitation (250 rpm).

All control and test flasks were sampled in the manner described previously at t=3, 6, and 24 hours and assayed on TSA plates as before.

In order to confirm that the antimicrobial solution was sufficiently neutralized by the D/E, a neutralization verification test was performed. A volume of 0.1 ml of the 2% molybdenum in PBS solution was placed into 0.9 ml of D/E neutralizing broth. The solution was mixed and then A. baumannii was added to a final concentration of approximately 1.0×10⁵ CFU/ml. The solution was mixed again and then was allowed to sit for ten minutes at room temperature (20.8° C.). Ten-fold serial dilutions of the neutralized solution were assayed as described previously.

Colonies were counted, and the levels of surviving A. baumannii CFU per ml in each flask determined. The data were reported as the logarithmic reduction using the formula −log₁₀ (Nₜ/N₀), where N₀ was the concentration of surviving A. baumannii at time=0 hours and Nₜ was the concentration of A. baumannii in the sample collected at time=t (e.g., 3, 6, or 24 hours).

A Student's t-test was used to statistically compare the reductions observed in the test flasks with the reductions observed in the control flasks (assuming unequal variances). The reductions in the test flasks were considered to be statistically significant if the resultant P value was ≤0.05.

### Results

The neutralization verification test results showed that the D/E neutralizing buffer was able to completely neutralize the antimicrobial effects of the 2% molybdenum solution. N₀ differences were observed between the samples neutralized with the D/E for 10 minutes prior to inoculation with A. baumannii and the control samples with A. baumannii inoculated into sterile PBS.

The results are shown in the table below. Small reductions were observed in the numbers of A. baumannii recovered from the control flasks at all of the exposure contact times (average of 0.55 log₁₀). In contrast, no A. baumannii were recovered from the test flasks amended with 2% molybdenum powder after 3, 6, or 24 hours of exposure. The bacterial numbers had fallen to below the detection limit of the assay (<5.0 CFU/ml); therefore, these reductions corresponded to a >4.61 log₁₀ reduction (>99.9975% reduction) and were highly statistically significant in comparison to the control flasks sampled at the same time (P=0.000013, P=0.00018, and P=0.0011, respectively).

The table below shows survival of Acinetobacter baumannii in phosphate buffered saline containing 2% pure molybdenum (wt %) after 3, 6, and 24 hours at 20.8° C.

| | | Control (PBS) | | | | |
|---|---|---|---|---|---|---|
| | | | Average | PBS with 2% Mo | | |
| Exposure | | | Log₁₀ | | Average Log₁₀ | Average |
| Time | | Log₁₀ | Reduction ± | Log₁₀ | Reduction ± | Percent |
| (hours) | Sample | Reduction* | SD | Reduction* | SD | Reduction |
| 3 | A | 0.55 | 0.57 ± | >4.61 | >4.61^{†} ± | >99.9975 |
| | B | 0.60 | 0.03 | >4.61 | 0.00 | |
| | C | 0.57 | | >4.61 | | |
| 6 | A | 0.58 | 0.66 ± | >4.61 | >4.61^{†} ± | >99.9975 |
| | B | 0.76 | 0.09 | >4.61 | 0.00 | |
| | C | 0.65 | | >4.61 | | |
| 24 | A | 0.17 | 0.43 ± | >4.61 | >4.61^{†} ± | >99.9975 |
| | B | 0.64 | 0.24 | >4.61 | 0.00 | |
| | C | 0.47 | | >4.61 | | |

*Initial Concentration = 2.03 × 10⁵ CFU/ml (t = 0 hours)
SD = standard deviation
> = the bacteria had fallen to below the detection limit of the assay (<5.0 CFU per milliliter or a 4.61 log₁₀ reduction); therefore, the reduction was >4.61 log₁₀ reduction (i.e., >99.9975% reduction).
^{†}Reduction was statistically significant (P ≤ 0.05) in comparison to the reductions observed on the control phosphate buffered saline flasks at the same exposure contact time.

### Example 6b—Suspension Time-Kill Test of Molybdenum Powder Suspended in Phosphate Buffered Saline Against Candida albicans

### Methods

A culture of Candida albicans (ATCC #10231) was prepared on the day before testing by inoculating one colony of the test organism into 100 ml of tryptic soy broth (TSB) and incubation overnight at 37° C.

On the test date, the yeast cells were washed by pelleting the cells via centrifugation. The supernatant was discarded and the pellet was re-suspended in 0.01 M phosphate-buffered saline (PBS; pH 7.4). Three washing steps were performed in total.

The cell suspension was diluted in 100 ml of sterile PBS in 250-ml screw cap Erlenmeyer flasks to obtain a density of ˜1×10⁵ colony-forming units (CFU) per ml. Six flasks were included in the experiment (3 control flasks, 3 test flasks). Test flasks contained 100 ml of PBS with 2 grams of pure molybdenum powder (2% Mo wt % solution). All six flasks were placed on an orbital shaker at room temperature 21.9° C. with agitation (200 rpm).

Samples from the three control flasks were collected immediately upon inoculation/mixing to determine the baseline yeast concentration at t=0 hours. A volume of 0.1 ml was removed from each and placed into separate 0.9-ml volumes of Dey-Engley (D/E) neutralizing broth. The samples were vortexed for 10 seconds and then 10-fold serially diluted in PBS. The various dilutions were inoculated onto potato dextrose agar (PDA) plates using the spread plate method. The plates were incubated for 48 hours at 37° C. and the colonies enumerated.

All control and test flasks were sampled in the manner described previously at t=3, 6, and 24 hours and assayed on PDA plates as before.

In order to confirm that the antimicrobial solution was sufficiently neutralized by the D/E, a neutralization verification test was performed. A volume of 0.1 ml of the 2% molybdenum in PBS solution was placed into 0.9 ml of D/E neutralizing broth. The solution was mixed and then C. albicans was added to a final concentration of approximately 1.0×10⁵ CFU/ml. The solution was mixed again and then was allowed to sit for 10 minutes at room temperature (21.9° C.). Ten-fold serial dilutions of the neutralized solution were assayed as described previously. If the solution was completely neutralized, it was expected that there would be no reduction in C. albicans numbers in comparison to the controls in PBS alone.

Colonies were counted, and the levels of surviving C. albicans CFU per ml in each flask determined. The data were reported as the logarithmic reduction using the formula −log₁₀ (Nₜ/N₀), where N₀ was the concentration of surviving C. albicans at time=0 hours and N t was the concentration of C. albicans in the sample collected at time=t (i.e., 3, 6, or 24 hours).

A Student's t-test was used to statistically compare the reductions observed in the test flasks with the reductions observed in the control flasks. The reductions in the test flasks were considered to be statistically significant if the resultant P value was <0.05.

### Results

The neutralization verification test results showed that the D/E neutralizing buffer was able to completely neutralize the antimicrobial effects of the 2% molybdenum solution. N₀ differences were observed between the samples neutralized with the D/E for 10 minutes prior to inoculation with C. albicans and the control samples with C. albicans inoculated into sterile PBS. Therefore, the reductions observed during the subsequent exposure tests may be considered accurate.

The results are shown in table below. Small reductions were observed in the numbers of C. albicans recovered from the control flasks at all of the exposure contact times (average of 0.09 log₁₀). In contrast, no C. albicans were recovered from the test flasks amended with 2% molybdenum powder after 3, 6, or 24 hours of exposure. The yeast numbers fell to below the detection limit of the assay (<5.0 CFU/ml); therefore, these reductions corresponded to a >4.15 log₁₀ reduction (>99.993% reduction) and were highly statistically significant in comparison to the control flasks sampled at the same time (P=6.4×10⁻⁸, P=7.0×10⁻⁸, and P=1.1×10⁻⁷, respectively).

The table below shows survival of Candida albicans (ATCC #10231) in phosphate buffered saline containing 2% pure molybdenum (wt %) after 3, 6, and 24 hours at 21.9° C.

| | | Control coupons (PBS) | | PBS with 2% Mo | | |
|---|---|---|---|---|---|---|
| | | | Average | | Average | |
| Exposure | | | Log₁₀ | | Log₁₀ | Average |
| Time | | Log₁₀ | Reduction ± | Log₁₀ | Reduction ± | Percent |
| (hours) | Sample | Reduction* | SD | Reduction* | SD | Reduction |
| 3 | A | 0.00 | 0.04 ± | >4.15 | >4.15^{†} ± | >99.993 |
| | B | 0.11 | 0.06 | >4.15 | 0.00 | |
| | C | 0.02 | | >4.15 | | |
| 6 | A | 0.00 | 0.06 ± | >4.15 | >4.15^{†} ± | >99.993 |
| | B | 0.03 | 0.07 | >4.15 | 0.00 | |
| | C | 0.14 | | >4.15 | | |
| 24 | A | 0.12 | 0.18 ± | >4.15 | >4.15^{†} ± | >99.993 |
| | B | 0.27 | 0.08 | >4.15 | 0.00 | |
| | C | 0.15 | | >4.15 | | |

*Initial Concentration = 7.00 × 10⁴ CFU/ml (t = 0 hours)
SD = standard deviation
> = the viable yeast had fallen to below the detection limit of the assay (<5.0 CFU per milliliter or a 4.15 log₁₀ reduction); therefore, the reduction was >4.15 log₁₀ reduction (i.e., >99.993% reduction).
^{†}Reduction was statistically significant (P ≤ 0.05) in comparison to the reductions observed on the control phosphate buffered saline flasks at the same exposure contact time.

### Example 7—X-Ray Diffraction Analysis Report

### Purpose

The present example provides an X-ray diffraction (XRD) analysis to determine certain crystalline phases present in a sample of molybdenum that has been activated using hydrogen peroxide (as reported herein in Examples above).

### Results

The sample was placed into a bulk sample holder and pressed flat with a glass slide for analysis. XRD data was collected by a coupled Theta:2-Theta scan on a Rigaku Ultima-III diffractometer equipped with Copper X-ray tube, Ni beta filter, parafocusing optics, computer-controlled slits, and D/tex Ultra 1D strip detector.

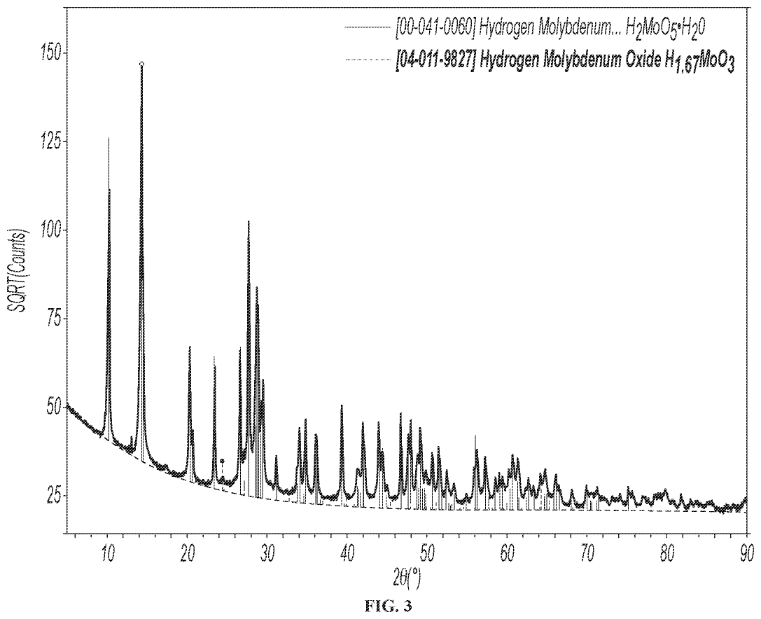

FIG. 3 shows the phase identification results for the sample obtained by comparing the background-subtracted experimental data to the ICDD/ICSD diffraction database. Intensity was plotted using square root (counts) to emphasize the weaker peaks. Monoclinic hydrogen molybdenum oxide hydrate (H2MoO5·H2O) was the primary phase observed in the sample with trace amounts of hydrogen molybdenum oxide (H1.67MoO3).

Semiquantitative analysis was performed using WPF (that is, whole pattern fitting), which is a subset of Rietveld Refinement that accounts for all intensity above the background curve. This technique requires that either the structure factors and atomic locations or the reference intensity ratio (a way of comparing the diffracting power of different phases) are known for all phases identified. In this case, quantitative analysis by XRD was not attempted for this sample because the reference intensity ratio (RIR), which is needed to account for the relative diffraction intensity from different crystal structures, is not available for the major phase and because the peak near 17 degrees remains unidentified.

### Example 8—Suspension Time-Kill Test of Molybdenum Powder Suspended in Phosphate Buffered Saline Against Escherichia coli

### Methods

A culture of Escherichia coli was prepared on the day before testing by inoculating one colony of the test organism into 100 ml of tryptic soy broth (TSB) and incubation overnight at 37° C.

To test, bacterial cells were washed by pelleting the cells via centrifugation. The supernatant was discarded and the pellet was re-suspended in 0.01 M phosphate-buffered saline (PBS; pH 7.4). Three washing steps were performed in total.

The cell suspension was diluted in 10 ml of sterile PBS in 50-ml conical tubes to obtain a density of ˜1×10⁶ colony-forming units (CFU) per ml. Six tubes were included in the experiment (3 control tubes, 3 test tubes).

Samples from the three control tubes were collected immediately upon inoculation/mixing to determine the baseline bacterial concentration at t=0 hours. A volume of 0.1 ml was removed from each and placed into separate 0.9-ml volumes of Dey-Engley (D/E) neutralizing broth. The samples were vortexed for 10 seconds and then 10-fold serially diluted in PBS. The various dilutions were inoculated onto eosin methylene blue (EMB) agar plates using the spread plate method. The plates were incubated for 24 hours at 37° C. and the colonies enumerated.

Two days prior to the experiment, 5.0 mg of pure molybdenum powder was added to each of the test conical tubes to allow for the powder to dissolve completely [resulting in 0.05% Mo solutions (wt %)]. At t=0 hours, all six tubes (control and test samples) were placed on an orbital shaker at a slant)(˜45° at room temperature 22.1° C. with agitation (250 rpm).

All control and test conical tubes were sampled in the manner described previously at t=3 and 24 hours and assayed on EMB plates as before.

In order to confirm that the antimicrobial solution was sufficiently neutralized by the D/E, a neutralization verification test was performed. A volume of 0.1 ml of the 0.05% molybdenum in PBS solution was placed into 0.9 ml of D/E neutralizing broth. The solution was mixed and then E. coli was added to a final concentration of approximately 1.0×10⁶ CFU/ml. The solution was mixed again and then was allowed to sit for ten minutes at room temperature (22.1° C.). Ten-fold serial dilutions of the neutralized solution were assayed as described previously.

Colonies were counted, and the levels of surviving E. coli CFU per ml in each tube determined. The data were reported as the logarithmic reduction using the formula −log₁₀ (Nₜ/N₀), where N₀ was the concentration of surviving E. coli at time=0 hours and N t was the concentration of E. coli in the sample collected at time=t (e.g., 3 or 24 hours).

A Student's t-test was used to statistically compare the reductions observed in the test conical tubes with the reductions observed in the control tubes. The reductions in the test conical tubes were considered to be statistically significant if the resultant P value was <0.05.

### Results

The neutralization verification test results showed that the D/E neutralizing buffer was able to completely neutralize the antimicrobial effects of the 0.05% molybdenum solution. N₀ differences were observed between the samples neutralized with the D/E for 10 minutes prior to inoculation with E. coli and the control samples with E. coli inoculated into sterile PBS.

The results are shown in the table below. Small reductions were observed in the numbers of E. coli recovered from the control tubes at all of the exposure contact times (average of 0.02 log₁₀). In contrast, fewer E. coli were recovered from the test conical tubes amended with 0.05% molybdenum powder after 3 hours of exposure (an average reduction of 2.09 log₁₀); no E. coli were recovered from the test conical tubes after 24 hours. Thus after 24 hours, the bacterial numbers had fallen to below the detection limit of the assay (<5.0 CFU/ml); these reductions corresponded to a >5.69 log₁₀ reduction (>99.9998% reduction). The reductions observed with the 0.05% Mo solutions after 3 and 24 hours were highly statistically significant in comparison to the control tubes sampled at the same time (P=0.03 and P=5.13×10⁻⁹, respectively).

The table below shows survival of Escherichia coli in phosphate buffered saline containing 0.05% pure molybdenum (wt %) after 3 and 24 hours at 22.1° C.

| | | Control coupons (PBS) | | PBS with 0.5% Mo | | |
|---|---|---|---|---|---|---|
| | | | Average | | Average | |
| Exposure | | | Log₁₀ | | Log₁₀ | Average |
| Time | | Log₁₀ | Reduction ± | Log₁₀ | Reduction ± | Percent |
| (hours) | Sample | Reduction* | SD | Reduction* | SD | Reduction |
| 3 | A | 0.00 | 0.01 ± | 1.51 | 2.09 ± | >99.19 |
| | B | 0.00 | 0.01 | 2.00 | 0.64 | |
| | C | 0.02 | | 2.77 | | |
| 24 | A | 0.10 | 0.04 ± | >5.69 | >5.69^{†} ± | >99.9998 |
| | B | 0.00 | 0.05 | >5.69 | 0.00 | |
| | C | 0.02 | | >5.69 | | |

*Initial Concentration = 2.47 × 10⁶ CFU/ml (t = 0 hours)
SD = standard deviation
> = the bacteria had fallen to below the detection limit of the assay (<5.0 CFU per milliliter or a 5.69 log₁₀ reduction); therefore, the reduction was >5.69 log₁₀ reduction (i.e., >99.9998% reduction).

1. An anti-pathogenic liquid composition comprising:
about 0.000000001% by weight to about 5% by weight of particles of an active component; and
water, saline, and/or a solvent,
wherein the active component is or comprises at least one activated metal.

2. An anti-pathogenic liquid composition comprising:
about 0.00001% by weight to about 5% by weight of particles of an active component; and
water, saline, and/or a solvent,
wherein the active component is or comprises at least one activated metal.

3. The anti-pathogenic liquid composition of claim 2, wherein the composition comprises about 0.001% by weight to about 1% by weight of particles of an active component.

4. The anti-pathogenic liquid composition of claim 2 or 3, wherein the composition comprises about 0.001% by weight to about 0.1% by weight of particles of an active component.

5. The anti-pathogenic liquid composition of any one of claims 1-4, wherein the composition comprises about 0.001% by weight to about 0.05% by weight of particles of an active component.

6. The anti-pathogenic liquid composition of any one of claims 1-5, wherein the composition comprises about 0.001% by weight to about 0.01% by weight of particles of an active component.

7. The anti-pathogenic liquid composition of claim 1, wherein the composition comprises about 0.00001% by weight to about 0.001% by weight of particles of an active component.

8. The anti-pathogenic liquid composition of claim 1, wherein the composition comprises about 0.000000001% by weight to about 0.001% by weight of particles of an active component.

9. The anti-pathogenic liquid composition of claim 1, 6, or 8, wherein the composition comprises about 0.00001% by weight to about 0.0001% by weight of particles of an active component.

10. The anti-pathogenic liquid composition of claim 1, 6, or 8, wherein the composition comprises about 0.0001% by weight to about 0.001% by weight of particles of an active component.

11. The anti-pathogenic liquid composition of claim 1, 6, or 8, wherein the composition comprises about 0.000000001% by weight to about 0.00000001% by weight of particles of an active component.

12. The anti-pathogenic liquid composition of claim 1, 6, or 8, wherein the composition comprises about 0.00000001% by weight to about 0.0000001% by weight of particles of an active component.

13. The anti-pathogenic liquid composition of claim 1, 6, or 8, wherein the composition comprises about 0.0000001% by weight to about 0.000001% by weight of particles of an active component.

14. The anti-pathogenic liquid composition of claim 1, 6, or 8, wherein the composition comprises about 0.000001% by weight to about 0.00001% by weight of particles of an active component.

15. The anti-pathogenic liquid composition of any one of claims 1-14, wherein the at least one activated metal is a transition metal or a transition metal oxide.

16. The anti-pathogenic liquid composition of any one of claims 1-15, wherein the at least one activated metal is selected from Mn, Mo, Zn, Cu, Au, Ag, or an oxide thereof.

17. The anti-pathogenic liquid composition of claim 16, wherein the at least one activated metal is Mo, Mo(IV), Mo(V) or Mo(VI), or an oxide thereof.

18. The anti-pathogenic liquid composition of claim 16, wherein the at least one activated metal is Mn, Mn(VII), Mn(VI), Mn(V), Mn(IV), Mn(III), Mn(II), Mn(I), or an oxide thereof.

19. The anti-pathogenic liquid composition of claim 16, wherein the at least one activated metal or oxide thereof is selected from Mn, MnO, Mn₃O₄, Mn₂O₃, MnO₂, MnO₃, Mn₂O₇, H₂MnO₄, HMnO₄, Mo, MoO₂, MoO₃, MoO₅, Mo₂O₆, H₂MoO₅, Zn, ZnO, Cu, Cu₂O, CuO, Au, AuO, Au₂O₃, Ag, and Ag₂O.

20. The anti-pathogenic liquid composition claim 19, wherein the at least one activated metal or oxide thereof is Mo, MoO₂, MoO₃, H₂MoO₅, or Mo₂O₆.

21. The anti-pathogenic liquid composition claim 19, wherein the at least one activated metal or oxide thereof is Mn, MnO, Mn₃O₄, Mn₂O₃, MnO₂, MnO₃, Mn₂O₇, H₂MnO₄, HMnO₄.

22. The anti-pathogenic liquid composition of any one of claims 1-21, wherein the at least one activated metal is Mo, or an oxide thereof, having a cubic, spherical, monoclinic, hexagonal, orthorhombic, tetragonal, triclinic, or rhombohedral crystal structure.

23. The anti-pathogenic liquid composition of any one of claims 1-22, wherein the pH of the anti-pathogenic liquid composition is about 5.5 or less.

24. The anti-pathogenic liquid composition of any one of claims 1-23, wherein the pH of the anti-pathogenic liquid composition is about 4.0 or less.

25. The anti-pathogenic liquid composition of any one of claims 1-24, wherein the pH of the anti-pathogenic liquid composition is about 2.0 or less.

26. The anti-pathogenic liquid composition of any one of claims 1-25, wherein the pH of the anti-pathogenic liquid composition is about 1.5 or less.

27. The anti-pathogenic liquid composition of any one of claims 1-22, wherein the pH of the anti-pathogenic liquid composition is about 7.

28. The anti-pathogenic liquid composition of any one of claims 1-22, wherein the pH of the anti-pathogenic liquid composition is about 7.5 or more.

29. The anti-pathogenic liquid composition of any one of claims 1-22 and 28, wherein the pH of the anti-pathogenic liquid composition is about 9 or more.

30. The anti-pathogenic liquid composition of any one of claims 1-22, 28, and 29, wherein the pH of the anti-pathogenic liquid composition is about 11.5 or more.

31. The anti-pathogenic liquid composition of any one of claims 1-22 and 28-30, wherein the pH of the anti-pathogenic liquid composition is about 14.

32. The anti-pathogenic liquid composition of any one of claims 1-31, wherein the particles of the at least one active metal are microparticles, wherein the microparticles have a size of about 1 μm to about 1000 μm.

33. The anti-pathogenic liquid composition of any one of claims 1-32, wherein the particles of the at least one active metal are microparticles, wherein the microparticles have a size of about 10 μm to about 85 μm.

34. The anti-pathogenic liquid composition of any one of claims 1-33, wherein the particles of the at least one active metal are microparticles, wherein the microparticles have a size of about 10 μm to about 50 μm.

35. The anti-pathogenic liquid composition of any one of claims 1-34, wherein the particles of the at least one active metal are microparticles, wherein the microparticles have a size of about 20 μm to about 50 μm.

36. The anti-pathogenic liquid composition of any one of claims 1-35, wherein the particles of the at least one active metal are microparticles, wherein the microparticles have a size of about 30 μm to about 50 μm.

37. The anti-pathogen liquid composition of any one of claims 1-36, wherein the particles of the at least one active metal are microparticles, wherein the microparticles have a size of about 40 μm to about 50 μm.

38. The anti-pathogenic liquid composition of any one of claims 1-37, wherein the particles of the at least one active metal are microparticles, wherein the microparticles have a size of about 40 μm to about 45 μm.

39. The anti-pathogenic liquid composition of any one of claims 1-31, wherein the particles of the at least one active metal are nanoparticles, wherein the nanoparticles have a size of about 1 nm to about 1000 nm.

40. The anti-pathogenic liquid composition of any one of claims 1-31 and 39, wherein the particles of the at least one active metal are nanoparticles, wherein the nanoparticles have a size of about 10 nm to about 85 nm.

41. The anti-pathogenic liquid composition of any one of claims 1-31, 39, and 40, wherein the particles of the at least one active metal are nanoparticles, wherein the nanoparticles have a size of about 10 nm to about 50 nm.

42. The anti-pathogenic liquid composition of any one of claims 1-31 and 39-41, wherein the particles of the at least one active metal are nanoparticles, wherein the nanoparticles have a size of about 20 nm to about 50 nm.

43. The anti-pathogenic liquid composition of any one of claims 1-31 and 39-42, wherein the particles of the at least one active metal are nanoparticles, wherein the nanoparticles have a size of about 30 nm to about 50 nm.

44. The anti-pathogen liquid composition of any one of claims 1-31 and 39-43, wherein the particles of the at least one active metal are nanoparticles, wherein the nanoparticles have a size of about 40 nm to about 50 nm.

45. The anti-pathogenic liquid composition of any one of claims 1-31 and 39-43, wherein the particles of the at least one active metal are nanoparticles, wherein the nanoparticles have a size of about 40 nm to about 45 nm.

46. The anti-pathogenic liquid composition of any one of claims 1-45, further comprising a second metal or metal oxide.

47. The anti-pathogenic liquid composition of claim 46, wherein the second metal is selected from Ni, Zn, Mn, Au, Ag, Cu, and Pd, or oxides thereof.

48. The anti-pathogenic liquid composition of claim 46 or 47, wherein the second metal is selected from Zn or ZnO.

49. The anti-pathogenic liquid composition of any one of claims 46-48, wherein the anti-pathogenic liquid composition comprises about 0.000000001% to about 5% by weight of the second metal.

50. The anti-pathogenic liquid composition of any one of claims 46-49, wherein the anti-pathogenic liquid composition comprises about 0.1% to about 5% by weight of the second metal.

51. The anti-pathogenic liquid composition of any one of claims 46-50, wherein the anti-pathogenic liquid composition comprises about 0.1% to about 3% by weight of the second metal.

52. The anti-pathogenic liquid composition of any one of claims 46-51, wherein the anti-pathogenic liquid composition comprises about 0.1% to about 1% by weight of the second metal.

53. The anti-pathogenic liquid composition of any one of claims 46-49, wherein the anti-pathogenic liquid composition comprises about 0.00001% to about 0.001% by weight of the second metal.

54. The anti-pathogenic liquid composition of any one of claims 46-49, wherein the anti-pathogenic liquid composition comprises about 0.001% to about 0.1% by weight of the second metal.

55. The anti-pathogenic liquid composition of any one of claims 46-49, wherein the anti-pathogenic liquid composition comprises about 0.000000001% to about 0.00000001% by weight of the second metal.

56. The anti-pathogenic liquid composition of any one of claims 46-49, wherein the anti-pathogenic liquid composition comprises about 0.00000001% to about 0.0000001% by weight of the second metal.

57. The anti-pathogenic liquid composition of any one of claims 46-49, wherein the anti-pathogenic liquid composition comprises about 0.000001% to about 0.00001% by weight of the second metal.

58. The anti-pathogenic liquid composition of any one of claims 46-49, wherein the anti-pathogenic liquid composition comprises about 0.00001% to about 0.0001% by weight of the second metal.

59. A method for neutralizing pathogens on a surface, the method comprising a step of contacting the surface with the anti-pathogenic liquid composition of any one of claims 1-58.

60. The method of claim 59, wherein the surface is human skin.

61. The method of claim 60, wherein the step of contacting human skin occurs prior to a surgical procedure.

62. The method of claim 60, wherein the step of contacting human skin occurs during a surgical procedure.

63. The method of claim 60, wherein the step of contacting human skin occurs after a surgical procedure.

64. The method of claims 59-63, wherein the pathogens are selected from Gram positive bacteria, Gram negative bacteria, fungi, viruses, and algae.

65. The method of any one of claims 59-63, wherein the pathogens are selected from micrococcus, staphylococcus, bacillus, pseudomonas, legionella, salmonella, listeria, Clostridium perfringens, Acinetobacter baumannii, Escherichia coli, coronaviruses, rhinoviruses, influenza, norovirus, adenovirus, parainfluenza, respiratory syncytial virus, and enterovirus.
